(19) United States
(12) Patent Application Publication
(10) Pub. No.: US 2026/0175196 A1
(43) Pub. Date: Jun. 25, 2026

(54) METHOD FOR SEQUESTERING METHIONINE CONTAINED IN A LIQUID AND MATERIAL SUITABLE FOR IMPLEMENTING SUCH A METHOD

(71) Applicants: UNIVERSITE DE BORDEAUX, BORDEAUX (FR); INSTITUT POLYTECHNIQUE DE BORDEAUX, TALENCE Cedex (FR); CENTRE NATIONAL DE LA RECHERCHE SCIENTIFIQUE, PARIS (FR)

(72) Inventors: Dario BASSANI, Pessac (FR); Amaury FURET, Bordeaux (FR); James FINDLAY, Dunedin (NZ)

(21) Appl. No.: 18/719,320

(22) PCT Filed: Dec. 13, 2022
(86) PCT No.: PCT/EP2022/085685

§ 371 Date: Jun. 13, 2024

(30) Foreign Application Priority Data
Dec. 16, 2021 (FR) FR2113725

(51) Int. Cl. B01J 20/285; C07C 323/58

(52) U.S. Cl. CPC B01J 20/285; C07C 323/58

(57) ABSTRACT

A method for sequestering methionine molecules contained in a liquid medium, comprising contacting this liquid medium with a solid polymer material comprising monomer units based on porphyrin containing copper. This method is particularly applicable to the processing of a liquid food product, such as wine, with a view to decreasing the risk of a lightstruck taste occurring.

The present invention lies in the field of the treatment of liquids, in particular beverages intended for human consumption.

More particularly, the present invention relates to a method for sequestering methionine molecules contained in a liquid medium.

The invention also relates to a more general method of treating a liquid food product in order to reduce the risk of apparition of a light-struck taste therein, as well as a method of detecting the presence of methionine in a liquid. The invention also relates to a particular polymer material suitable for implementing such methods, capable of sequestering methionine.

### CONTEXT OF THE INVENTION

In the present description, the term “wine” encompasses both still wines and effervescent wines.

Wines are products consumed worldwide, and particular attention is given to their quality. Numerous defects are likely to occur therein, whether during production or during storage thereof. The aromatic deviation the most widely known to the general public is the cap taste, which manifests itself when opening the bottle. Another defect that is just as deleterious, referred to as “light-struck taste”, can develop as a result of exposure of the wine to light. This organoleptic alteration leads to the formation in the wine of a taste similar to that of baked cabbages or of a wet wool odor. This defect may also be present in the products derived from the malting industry or from the dairy industry.

In wines, the light-struck taste is formed following the photo-oxidation reaction of the sulfur-containing amino acids, which are produced therein during alcoholic fermentation, by a particular photosensitizer, which is present therein naturally: riboflavin. When riboflavin absorbs light, at a wavelength between 300 and 500 nm, a singlet excited state is formed. This state is predominantly transformed into a triplet state by an inter-system crossing and it can then oxidize, by electron transfer, the sulfur-containing amino acids, and quite particularly methionine. Thus, these amino acids are degraded into volatile sulfur-containing products such as methanethiol, dimethyldisulfide and dimethyltrisulfide. These sulfur-containing species have very low perception thresholds, and concentrations in the range of micromoles lead to the formation of a light-struck taste that is detectable by consumers by tasting. The product can develop this defect very rapidly. For example, the exposure of an effervescent wine flute to sunlight for 15 minutes is enough to form the light-struck taste therein. In terms of taste, the product, and this is particularly the case for white and rose wines and for effervescent wines, develops a pronounced bitterness.

In dairy products, the light-struck taste is also primarily formed following the degradation of sulfur-containing amino acids, in particular methionine, as a consequence of the reaction thereof with riboflavin in the triplet state. This reaction leads to the formation of malodorous volatile sulfur-containing compounds such as methanethiol and dimethyldisulfide, responsible for the apparition of the light-struck taste.

In order to avoid the apparition of a light-struck taste in these liquid products, it is necessary to keep them permanently protected from light, from production until consumption thereof. This proves to be difficult to achieve in practice. In particular, the opaque glasses used for conditioning wines, in particular effervescent wines, do not have light filtering properties that are good enough to effectively prevent the apparition of a light-struck taste, or are very expensive to implement in the context of industrial manufacture. Furthermore, the use of highly opaque packages, which hide the product, is disadvantageous for sale.

In order to limit the risk of the apparition of a light-struck taste in wines, it has been proposed in the prior art to reduce the concentration of riboflavin therein by different oenological approaches, more particularly either by limiting release thereof by the yeasts during the wine preparation process, for example by implementing, for the alcoholic fermentation, strains of Saccharomyces producing little riboflavin, or by extracting the latter from the wine by means of different inorganic adsorbents, such as activated carbon (Fracassetti et al., Australian Journal of Grape and Wine Research 23, 329-333, 2017). However, the decrease in the concentration of riboflavin in wines results in a fading of its coloration. Furthermore, processing wine with adsorbents such as activated carbon can also lead to a reduction in the concentration of the other aromatic compounds of wine, in particular polyphenols. Hence, such a processing would lead to a modification of the aromatic profile of the wine and the deterioration of its organoleptic properties.

Thus, at the present time, apart from the use of a very opaque package, which is not very suitable for numerous beverages intended for human consumption, there is no satisfactory solution for protecting food liquids, in particular wines and dairy products, from the apparition of a light-struck taste, without modifying their sensory profile.

While searching for a solution to prevent the apparition of a light-struck taste of liquid products containing sulfur-containing amino acids and riboflavin, due to the formation of volatile sulfur-containing compounds upon exposure to light, the present inventors have sought to extract from these products, not riboflavin, as has been proposed by the prior art, but methionine, a sulfur-containing amino acid primarily at the origin, with riboflavin, of the formation of these deleterious volatile sulfur-containing compounds.

The present inventors have discovered that particular polymer materials make it possible to efficiently sequester the methionine molecules contained in liquid media, and removing them therefrom, and they have observed that the apparition of a light-struck taste is then significantly reduced when these liquid media are exposed afterwards to light in the wavelength range likely to excite riboflavin. This inhibition of the apparition of a light-struck taste is furthermore obtained without modifying the organoleptic properties of the liquid medium, which proves to be highly advantageous in the context of beverages intended for human consumption.

Thus, the present invention aims to overcome the drawbacks of the solutions proposed by the prior art to prevent the apparition of a light-struck taste in beverages, in particular the drawbacks set out hereinbefore, by proposing a method that allows effectively preventing such an apparition, yet without modifying the sensory profile of the beverage. More generally, the present invention aims to provide a method for effectively sequestering methionine molecules contained in a liquid medium.

Additional objectives of the invention are for this method to simple to implement, and at low cost.

### SUMMARY OF THE INVENTION

Thus, according to a first aspect, according to the present invention, there is provided a method for sequestering methionine molecules contained in a liquid medium, comprising contacting this liquid medium with solid polymer material comprising monomer units based on porphyrin containing copper.

In preferred embodiments of the invention, the solid polymer material contains at least one hydrophilic group, preferably at least one ionic group, preferably at least one anionic group, more preferably at least one sulfonate ion group or a salt thereof. In particular, this group may be borne by at least one of said monomer units based on porphyrin containing copper of the polymer, preferably by each of these monomer units.

A polymer that is particularly suitable for the implementation of this method corresponds to the general formula (V):

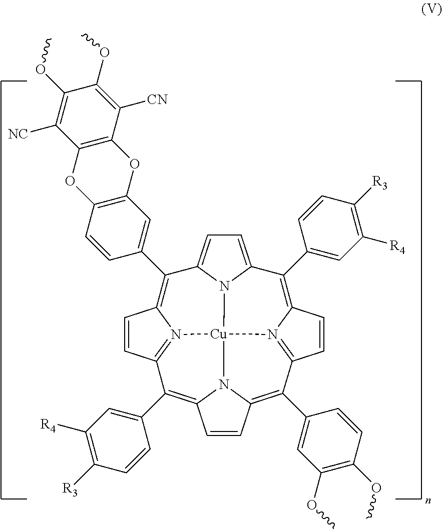

wherein:
R₃ and R₄, identical or different, each represent a hydrogen atom or a group selected from among a sulfonate ion or a salt thereof, a carboxylate ion or a salt thereof, an ammonium ion or a salt thereof, or a boronic acid group, and n is an integer greater than 2, and preferably less than 1000.

Another polymer that is particularly suitable for implementing this method is such that it can be obtained by a synthesis method comprising steps of:
a/ preparing a porphyrin derivative of formula (VI):

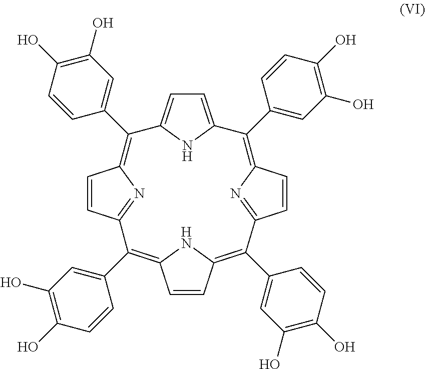 [SMILES: Oc1ccc(-c2c3nc(c(-c4ccc(O)c(O)c4)c4ccc([nH]4)c(-c4ccc(O)c(O)c4)c4nc(c(-c5ccc(O)c(O)c5)c5ccc2[nH]5)C=C4)C=C3)cc1O]

b/ polycondensing the porphyrin derivative of formula (VI) with a compound of formula (VII):

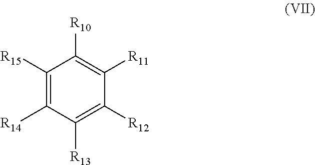

wherein:
R₁₀ and R₁₁ each represent a carbonitrile group —CN, and R₁₁, R₁₂, R₁₄ and R₁₅, identical or different, each represent a halogen atom, in particular a fluorine atom, in the presence of a compound of formula (VIII):

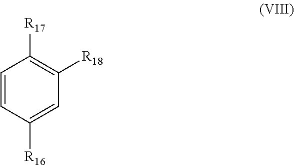

wherein:
R₁₆ represents a hydroxyl, primary or secondary amine or thiol group,
and R₁₇ and R₁₈, identical or different, each represent a hydrogen atom or a group selected from among a sulfonate ion or a salt thereof, a carboxylate ion or a salt thereof, an ammonium ion or a salt thereof, or a boronic acid group, so as to form a polymer whose some monomer units contain a porphyrin ring, and
c/ complexing the porphyrin rings of the polymer obtained in step b/ with copper.

According to another aspect, the present invention relates to a method of treating a liquid food product, in particular a beverage intended for human consumption, in order to reduce, and even eliminate, the risk of apparition of a light-struck taste, this method comprising the implementation, on this liquid food product, of a method for sequestering methionine molecules contained in a liquid medium according to the invention, then, after a contact time of at least 30 minutes, preferably of at least 2 hours, in particular between 2 hours and 24 months, for example between 4 hours and 24 months, the separation of the polymer material and of the liquid food product.

In particular, this food product may be selected from among wines, in particular white wines, rose wines and effervescent wines, dairy products and malting products, and more generally all liquid food products containing methionine likely to be degraded.

Another aspect of the invention is a method of detecting the presence of methionine in a liquid, which comprises the implementation, on a sample of this liquid, of a method for sequestering methionine molecules contained in a liquid medium according to the invention, then, after a time of contact of at least 30 minutes, the separation of the polymer material and the liquid and the analysis of the polymer material thus recovered for the presence of methionine.

The invention also relates to a porous solid polymer material particularly suitable for the implementation of the methods according to the invention. This polymer material is such that it can be obtained by a synthesis method comprising steps of:
a/ preparing a porphyrin derivative of formula (VI):

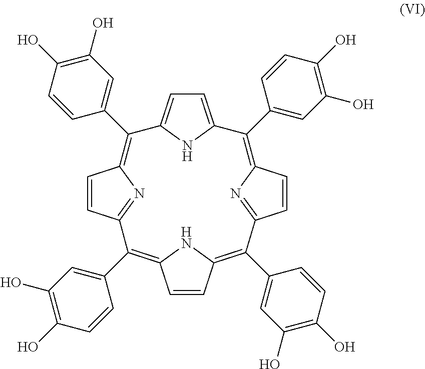 [SMILES: Oc1ccc(-c2c3nc(c(-c4ccc(O)c(O)c4)c4ccc([nH]4)c(-c4ccc(O)c(O)c4)c4nc(c(-c5ccc(O)c(O)c5)c5ccc2[nH]5)C=C4)C=C3)cc1O]

b/ polycondensing the porphyrin derivative of formula (VI) with a compound of formula (VII):

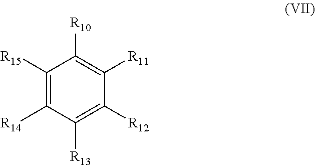

wherein:
R₁₀ and R₁₃ each represent a carbonitrile group —CN,
And R₁₁, R₁₂, R₁₄ and R₁₅, identical or different, each represent a halogen atom, in particular a fluorine atom, in the presence of a compound of formula (VIII):

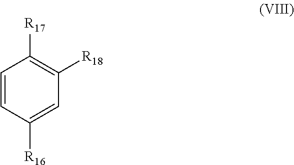

wherein:
R₁₆ represents a hydroxyl, primary or secondary amine or thiol group,
and R₁₇ and R₁₈, identical or different, each represent a hydrogen atom or a group selected from among a sulfonate ion or a salt thereof, a carboxylate ion or a salt thereof, an ammonium ion or a salt thereof, or a boronic acid group, so as to form a polymer whose some monomer units are based on a porphyrin ring, and
c/ complexing the porphyrin rings of the polymer obtained in step b/ with copper.

### BRIEF DESCRIPTION OF THE FIGURES

FIG. 1 illustrates an example of a reaction scheme for the synthesis of a linear polymer that can be implemented in accordance with the invention.

FIG. 2 illustrates an example of a reaction scheme for the synthesis of a crosslinked polymer that can be implemented in accordance with the invention.

FIG. 3 shows the ¹H NMR spectrum of methionine in deuterium oxide D₂O with the identification of each of the obtained signals.

FIG. 4 shows graphs representing the intensity of the proton NMR signals of methionine (signals 1, 2 and 4 identified in FIG. 3) as a function of the mass equivalents of repeat units of the polymer mixed with methionine, in a/ for the linear polymer PL1 in accordance with the invention, in b/ for the linear polymer PL2 in accordance with the invention and in c/ for the crosslinked polymer PR1 in accordance with the invention.

FIG. 5 shows a graph representing the intensity of the proton NMR signals of methionine (signals 1, 2 and 4 identified in FIG. 3) as a function of the mass equivalents of repeat units of the polymer mixed with methionine, for the crosslinked polymer PComp1 not in accordance with the invention.

FIG. 6 shows a graph representing the absorbance measured as a function of the concentration of fluorescent methionine derivative mixed with a crosslinked polymer in accordance with the invention PR1.

FIG. 7 shows a graph showing the total concentration of methanethiol (MeSH) and dimethyl disulfide (DMDS) in a sample of effervescent wine having been subjected to irradiation after having been brought into contact with a polymer material according to the invention, as a function of the concentration of polymer material in the sample—the dotted line represents the threshold of perception of a light-struck taste by a sensory panel on a sample of this same wine irradiated and not brought into contact with the polymer material.

### DETAILED DESCRIPTION OF THE INVENTION

The method for sequestering methionine molecules contained in a liquid medium according to the invention uses a solid polymer material based on organometallic type monomer units, more particularly containing a porphyrin ring, modified or not to bear one or more substituent(s), complexed with a copper atom.

Advantageously, this polymer material is selected so as not to be soluble in the liquid medium in which it is desired to sequester the methionine molecules. In particular, the porphyrin ring-based polymers recommended by the invention are advantageously insoluble in aqueous solutions and in hydroalcoholic solutions. Thus, it is possible to separate them easily from the liquid medium, without them leaving residues therein, at least in significant amounts, even after a long contact time with the liquid, for example as long as throughout the maturation of a wine in barrel.

Placed in contact with the methionine molecules contained in the liquid medium, the polymer material according to the invention advantageously has a particularly high adsorption capacity for these molecules, resulting, on the one hand, from the strong coordination bond forming between the copper atoms and the sulfur atoms of the methionine molecules, and, on the other hand, from the structure of the porphyrin rings itself. Surprisingly, this adsorption capacity is in particular much higher than that of other polymers of similar structure also in the form of complexes with copper.

Besides its high capacity for binding to the sulfur atom of methionine, copper offers the advantages of great abundance, reasonable price, and low toxicity for living organisms. For example, the maximum threshold of copper allowed in the wines is high, equal to 1 mg/L. Thus, in the case where the liquid medium is a beverage, bringing the latter into contact with the material in accordance with the invention based on copper does not generate therein, after separation thereof from the polymer material, any toxicity likely to prevent a subsequent consumption thereof.

Furthermore, the polymer material used according to the invention advantageously has a high selectivity with respect to methionine. In particular, when the liquid medium is a beverage, this material does not adsorb any compound contributing to the properties of the latter, and in particular to its sensory profile.

Preferably, the implemented solid polymer material is porous, preferably with a high degree of porosity, corresponding to a high surface/volume ratio. Such a feature increases even more the ability to trap the methionine molecules of the polymer material.

The contacting of the liquid medium with the solid polymer material can be carried out according to any method conventional per se for contacting a solid with a liquid. In particular implementations of the invention, the contacting of the liquid medium and the solid polymer material is carried out by incorporating the material into the liquid medium, and maintaining the material in the medium, preferably under stirring, for a contact time long enough to enable the establishment of bonds between the polymer material and the methionine molecules contained in the liquid medium. This contact time, which may vary according to the targeted particular application, may in particular be of at least 30 minutes.

The method according to the invention may further meet one or more of the features described hereinafter, implemented separately or in any of their technically-feasible combinations.

In particular, the exact characteristics of the solid polymer material are selected according to the intended application for the liquid medium after the methionine molecules have been extracted therefrom by means of the method according to the invention. When the method of the invention is intended for treating food liquids, these characteristics are thus selected so as to meet the regulatory requirements applied to compounds in contact with food products.

Preferably, the polymer material used according to the invention comprises at least three porphyrin monomer units containing copper. Preferably, the exact number of such monomer units is selected so as to ensure that the polymer material is insoluble in the liquid medium. It is within the skills of a person skilled in the art to determine this minimum number of monomer units according to the considered particular liquid medium.

In particular implementations of the invention, the polymer contains at least one hydrophilic group, which advantageously increases its compatibility with aqueous media.

Preferably, this hydrophilic group is an ionic group, and preferably an anionic group. Such a feature advantageously promotes trapping of methionine molecules at the surface of the polymer material, by formation of an ionic bond between the anionic groups borne by the polymer material and the primary amine groups of the methionine molecules. In preferred embodiments of the invention, the polymer contains at least one group selected from among the sulfonate ion group or a salt thereof, the carboxylate ion group or a salt thereof, the ammonium ion group or a salt thereof, or a boronic acid group, the sulfonate ion group and its salts being particularly preferred in the context of the invention.

In particular implementations of the invention, at least one such hydrophilic group is preferably borne by at least one of said monomer units based on copper-containing porphyrin, in particular at the porphyrin unit, preferably by several ones of these monomer units, and preferably each of them.

In particular implementations of the invention, the porphyrin ring of at least one of said monomer units, preferably several ones of these monomer units, and preferably all of them, is modified to bear at least one ionic group, preferably anionic, or one of its salts, preferably at least two ionic groups, preferably anionic groups, or one of their salts, the ionic groups borne by the same porphyrin ring being then possibly identical or different.

Alternatively, at least one such hydrophilic group may be borne by a crosslinking agent used for the preparation of the polymer material and being integrated in the structure of the latter.

When the hydrophilic group is a salt of an ionic group, this salt is preferably selected so as to be compatible with use in contact with food. When the ionic group is an anionic group, it is preferably an alkali metal salt, in particular a sodium salt.

Preferably, at least one anionic group borne by the polymer material, for example by a porphyrin ring, preferably each of the anionic groups borne by the polymer material, for example by the porphyrin rings, is a sulfonate ion group, where appropriate in the form of a salt, in particular an alkali metal salt and for example a sodium salt. Such a sulfonate ion group is particularly suitable for forming an ionic bond with the primary amine groups of methionine molecules at acidic pH. It is also suitable for application in contact with food.

In the embodiments of the invention wherein one or more ionic group(s) is/are borne by the porphyrin rings of the polymer, these ionic groups may be bound directly thereto, or via a spacer arm. Such a spacer arm then preferably includes a phenyl ring, on which the ionic group(s), in particular the sulfonate group(s), are then preferably directly linked.

In particular implementations of the invention, each of the porphyrin rings of the polymer is modified so as to bear one or more phenylsulfonate group(s). A sulfonate group is then preferably present on the phenyl ring at the meta position and/or at the para position, with respect to the porphyrin ring.

Preferably, the porphyrin rings of the polymer material do not bear a polyethylene glycol type group as the sole substituent. More generally, they can be free of any polyethylene glycol type group.

In particular implementations of the invention, in the polymer, at least one of the monomer units, preferably a plurality of these monomer units, and in particular all of these monomer units, corresponds to the general formula (I):

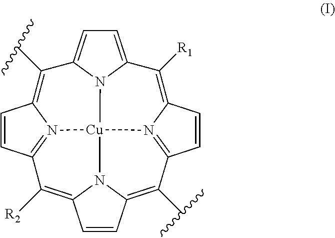

wherein R₁ and R₂, identical or different, each represent a hydrogen atom or a group of formula (II):

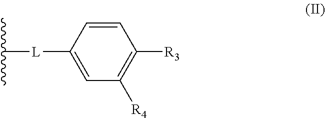

wherein:
R₃ and R₄, identical or different, each represent a hydrogen atom or a group selected from among a sulfonate ion or a salt thereof, a carboxylate ion or a salt thereof, an ammonium ion or a salt thereof, or a boronic acid group, preferably a sulfonate ion or an alkali metal salt, for example a calcium salt, of a sulfonate ion,
and L represents a covalent bond or a spacer arm.

Preferably, in formula (II), L represents a spacer arm of formula (III):

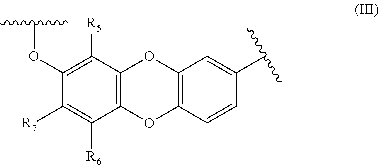

wherein:
R₅ and R₆ each represent a carbonitrile group —CN,
R₇ represents a halogen atom, in particular a fluorine atom.

In particular, such a spacer arm has the advantage of being rigid and of leading spontaneously to the formation of a porous structure.

In formula (III), the oxygen atom bound to the unit

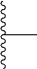

is preferably linked to the phenyl ring of the group of formula (II), and the phenyl ring bound to the unit

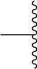

is preferably linked to the porphyrin ring. Such a polymer has a particularly significant methionine-specific adsorption capacity. This adsorption capacity is even greater when, in formula (II), at least R₃ or R₄ represents a sulfonate ion group, where appropriate in the form of a salt.

The bonding of the monomer units within the polymer may be realized by means of any crosslinking agent conventional per se.

For example, such a crosslinking agent may be of the type having two carboxylate groups.

In particular implementations of the invention, the crosslinking agent is such that the monomer units within the polymer are linked by a bonding group of formula (IV):

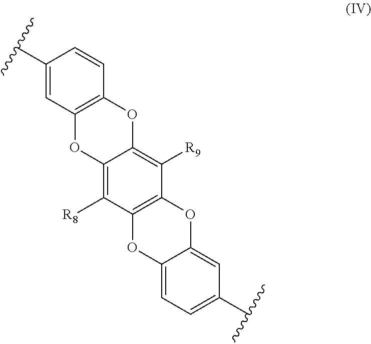

wherein R₈ and R₉ each represent a carbonitrile group —CN. The polymer used according to the invention may be of the linear type. It then offers the advantage of a perfectly controlled structure.

In particular, the polymer used according to the invention may have formula (V):

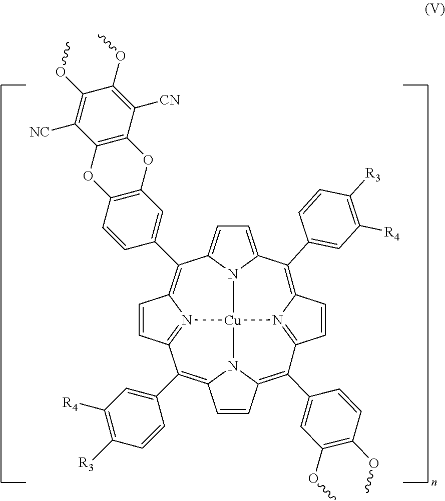

wherein:
R₃ and R₄, identical or different, each represent a hydrogen atom or a group selected from among a sulfonate ion or a salt thereof, a carboxylate ion or a salt thereof, an ammonium ion or a salt thereof, or a boronic acid group, the sulfonate ion and its salts, preferably an alkali metal salt, for example the sodium salt, being particularly preferred in the context of the invention,
and n is an integer greater than 2, and preferably less than 1,000.

Preferably, in formula (V), R₃ represents a hydrogen atom and R₄ represents a sulfonate ion or a salt thereof, preferably an alkali metal salt, for example a sodium salt. The polymer used according to the invention may then have formula (Va):

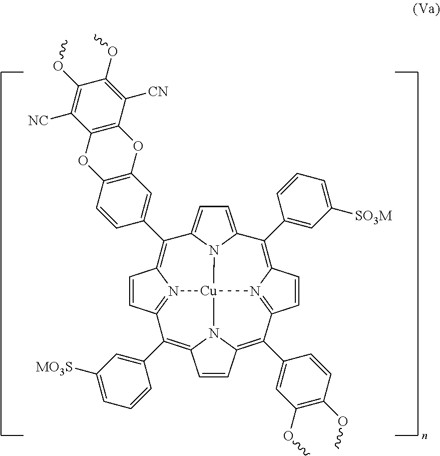

wherein:
M represents an alkali metal, in particular sodium, and
n is an integer greater than 2, and preferably less than 1,000.

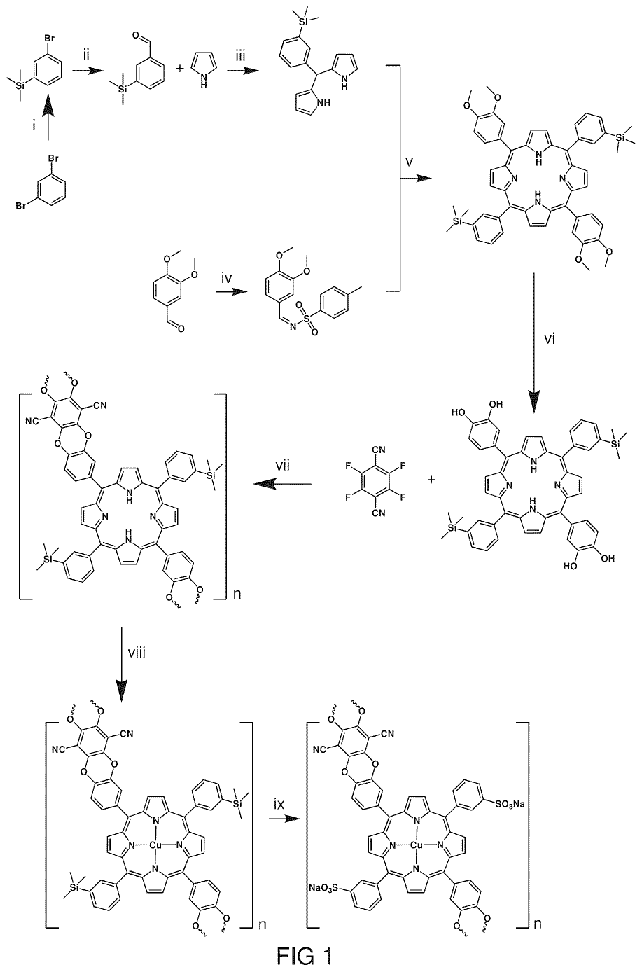

Such a linear polymer may be prepared according to any reaction scheme, the determination of which is within the skills of a person skilled in the art, for example according to the reaction scheme shown in FIG. 1, for the case in which M represents a sodium atom. Each of steps i to ix of this reaction scheme may be carried out in any manner conventional per se for a person skilled in the art. An example of operating conditions is given as example hereinafter in the present description.

In particularly preferred implementations of the invention, the polymer is a crosslinked polymer. Advantageously, in particular thanks to its intrinsic porosity, such a polymer has a methionine adsorption capacity greater than that of linear polymers.

Such a polymer in accordance with the invention can be obtained by a synthesis method comprising steps of:
a/ preparing a porphyrin derivative of formula (VI):

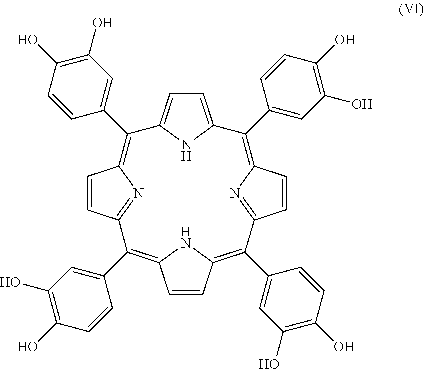 [SMILES: Oc1ccc(-c2c3nc(c(-c4ccc(O)c(O)c4)c4ccc([nH]4)c(-c4ccc(O)c(O)c4)c4nc(c(-c5ccc(O)c(O)c5)c5ccc2[nH]5)C=C4)C=C3)cc1O]

b/ polycondensing the porphyrin derivative of formula (VI) with a crosslinking agent, and
c/ complexing the porphyrin rings of the polymer obtained in step b/ with copper.

A particularly advantageous polymer in the context of the invention can be obtained by a synthesis method comprising steps of:
a/ preparing a porphyrin derivative of formula (VI):

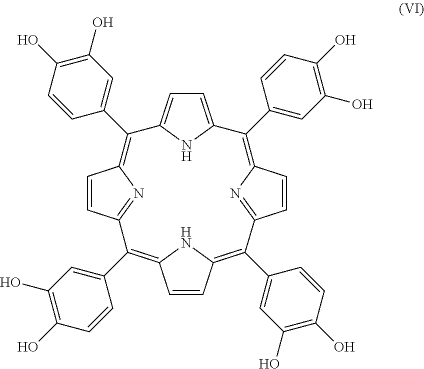 [SMILES: Oc1ccc(-c2c3nc(c(-c4ccc(O)c(O)c4)c4ccc([nH]4)c(-c4ccc(O)c(O)c4)c4nc(c(-c5ccc(O)c(O)c5)c5ccc2[nH]5)C=C4)C=C3)cc1O]

b/ polycondensing the porphyrin derivative of formula (VI) with a compound of formula (VII):

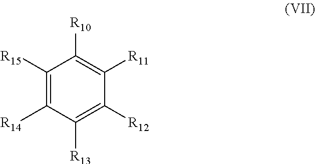

wherein:
R₁₀ and R₁₃ each represent a carbonitrile group —CN,
R₁₁, R₁₂, R₁₄ and R₁₅, identical or different, each represent a halogen atom, in particular a fluorine atom,
in the presence of a compound of formula (VIII):

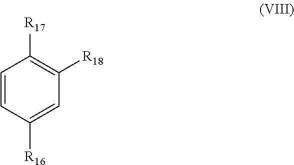

wherein:
R₁₆ represents a hydroxyl, primary or secondary amine or thiol group,
R₁₇ and R₁₈, identical or different, each represent a hydrogen atom or a group selected from among a sulfonate ion or a salt thereof, a carboxylate ion or a salt thereof, an ammonium ion or a salt thereof, or a boronic acid group, the sulfonate ion and its salts, preferably an alkali metal salt and for example the sodium salt, being particularly preferred according to the invention, so as to form a crosslinked polymer with random branches, and
c/ complexing the porphyrin rings of the polymer obtained in step b/ with copper.

Such a method has the advantage of a reduced number of steps, and of great ease and speed of implementation. Furthermore, it advantageously allows forming polymers with a high level of intrinsic porosity, the degree of crosslinking being in particular easily controllable by controlling the respective proportions of the compounds of formulas (VI), (VII) and (VIII) during the polycondensation step b/ of the method.

Each of the steps of the method for synthesizing the polymer may be carried out according to operating conditions conventional per se for a person skilled in the art.

Step a/ of preparing the porphyrin derivative of formula (VI) can for example be carried out by a first step a1/ of condensing 3,4-dimethoxybenzaldehyde with pyrrole, for example in propanoic acid under reflux, to form a first porphyrin derivative of formula (VI′):

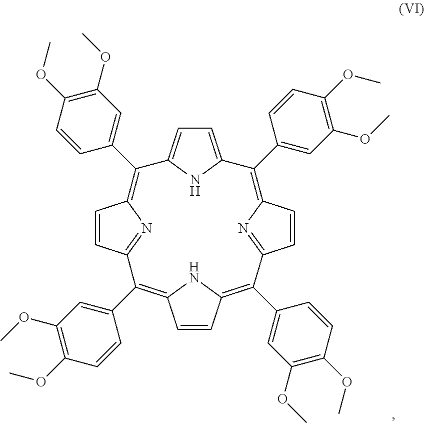

Afterwards, this compound of formula (VI′) may, after a possible purification, be subjected to a step a2/ of demethylation, for example by treatment with bromine tribromide, or a mixture of hydrobromic acid and acetic acid under reflux, in order to obtain the porphyrin derivative of formula (VI).

Step b/ of polycondensing the porphyrin derivative of formula (VI) with the compound of formula (VII), in the presence of the compound of formula (VIII), resulting in the formation of a random crosslinked polymer, containing end R₁₇, R18 groups, may for example be carried out by bringing together the different reagents and an excess of sodium carbonate. For example, the molar ratio of the different compounds of respective formulas (VI)/(VII)/(VIII) may be equal to 1/3/2.

Step c/ of the synthesis method, of metalation of the polymer obtained upon completion of step b/, may for example be carried out by bringing this polymer into contact with an excess of hydrated copper diacetate Cu(OAc)₂.

The crosslinked polymer obtained upon completion of this synthesis method is a random type one. Its exact structure cannot be determined, yet it is possible to propose the following formula (IX) for the structure of an average repeat unit of this polymer:

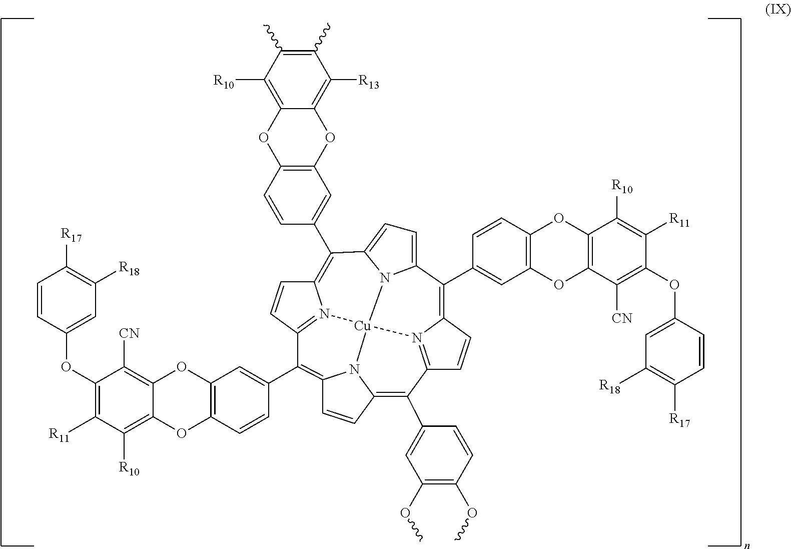

wherein
R₁₀, R₁₁, R₁₃, R₁₇, R₁₈ are as defined hereinbefore, and n is an integer greater than 2, and preferably less than 1,000.

In particular implementations of the invention:
in formula (VII), R₁₀ and R₁₃ each represent a carbonitrile group —CN and R₁₁, R₁₂, R₁₄ and R₁₅ each represent a fluorine atom, the compound of formula (VII) then being 2,3,5,6-tetrafluoroterephthalonitrile of formula (VIIa):

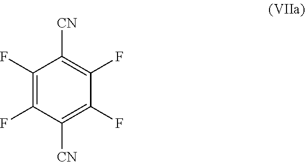 [SMILES: N#Cc1c(F)c(F)c(C#N)c(F)c1F]

and/or, in formula (VIII), R₁₆ represents a hydroxyl group, R₁₇ represents a sulfonate salt, preferably an alkali metal salt, for example a sodium salt, and R₁₈ represents a hydrogen atom, the compound of formula (VIII) then being phenolsulfonate, preferably of an alkali metal, in particular sodium, of formula (VIIIa):

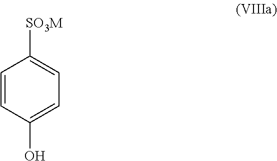

wherein M represents an alkali metal atom, for example a sodium atom.

It is then possible to propose, for the random crosslinked polymer thus obtained, the following formula (IXa) for the structure of an average repeat unit of this polymer:

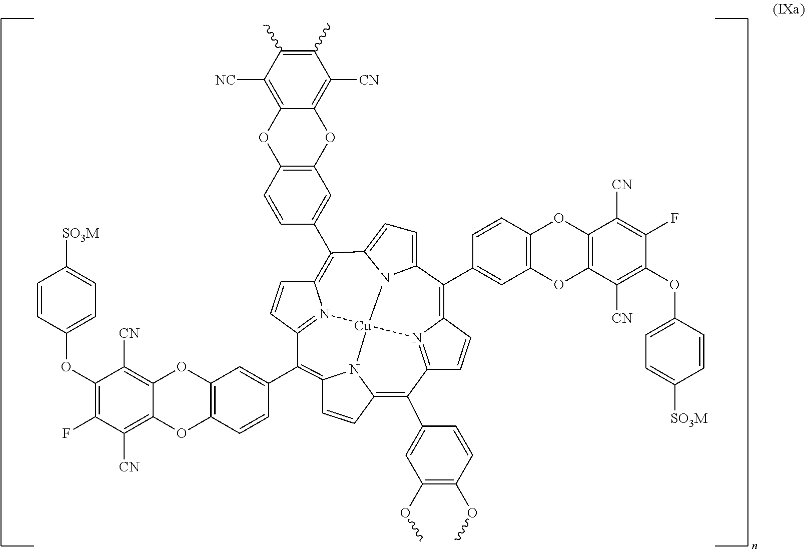

wherein:
M represents an alkali metal, for example sodium, and n is an integer greater than 2, and preferably less than 1,000.

Such a polymer material has a particularly strong and selective methionine sequestration capacity. In particular, the constant of association thereof with methionine is about 3×10⁶ M⁻¹. Its methionine adsorption capacity is estimated at about 250 mg/g of polymer material. Mechanisms underlying such a performance will not be prejudged here. However, it can be assumed that the high degree of porosity of the material, the bond forming between the copper (II) contained in the polymer and the sulfur atom of methionine, and the electrostatic interactions occurring between the sulfonate ion groups borne by the polymer and the primary amine groups of methionine, contribute thereto in combination.

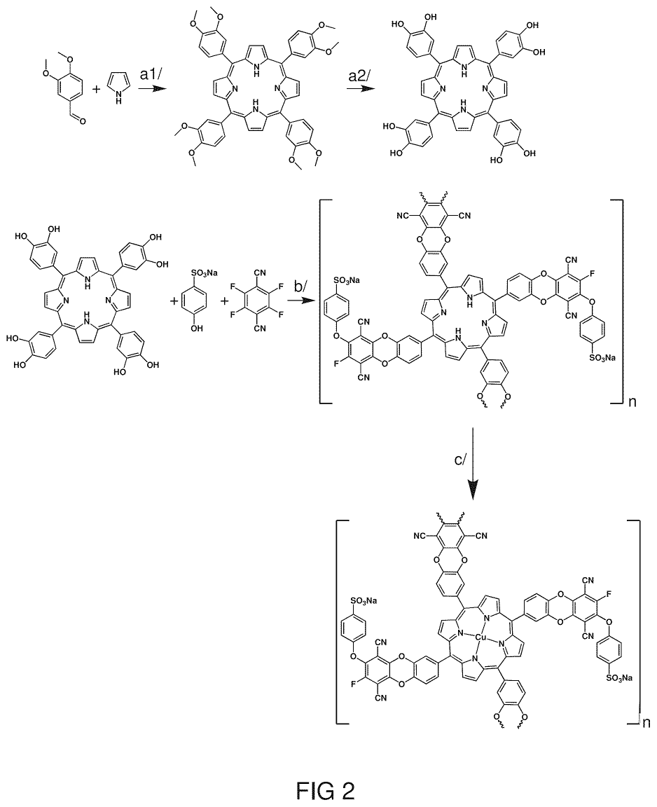

An example of a reaction scheme allowing obtaining such a crosslinked polymer material is shown in FIG. 2, for the case in which the sulfonate salt is a sodium salt.

In particular implementations of the invention, the polymer material is introduced into the liquid medium containing methionine, or likely to contain methionine, at a dose which depends on the amount of methionine to be sequestered, and which may for example be comprised between 0.01 and 50 g/l, in particular between 0.1 and 10 g/l, and more particularly between 0.2 and 2 g/l. For example, when the liquid medium is an effervescent wine, in particular during production thereof, in the alcoholic fermentation phase, the dose of polymer material introduced into the liquid medium may be about 0.2 g/l, or more.

Preferably, bringing the liquid medium and the polymer material into contact is carried out under stirring.

Preferably, the method according to the invention comprises, after a contact time enough to enable trapping of the methionine molecules by the polymer material, a step of separating the liquid medium and the polymer material sequestering methionine.

This separation step may be carried out by any solid-liquid separation method known to a person skilled in the art, for example by filtration, decantation, scouring during sparkling of effervescent wines, etc. After separation of the liquid medium, the polymer material may advantageously be regenerated for subsequent reuse. Thus, for this purpose, the method according to the invention may comprise a final step of separating the methionine molecules and the polymer material.

For example, such a step may consist in processing the polymer material with an alkaline washing solution, for example at a pH of 10, optionally assisted by ultrasonication. After separation of the solid polymer material from the washing solution, and rinsing(s) with water, a regenerated polymer material is obtained, having a high methionine adsorption capacity again.

The method for sequestering methionine molecules contained in a liquid medium finds application in many fields.

An application for which it proves to be particularly suitable is that of treating food liquids, in particular intended for human consumption, in order to avoid the apparition of a light-struck taste in the latter following exposure thereof to light.

Thus, one aspect of the invention relates to a method of treating a liquid food product, in particular a beverage intended for human consumption, in order to reduce, and even completely eliminate, the risk of apparition of a light-struck taste therein. This method comprises the implementation, on this liquid food product, of a method for sequestering methionine molecules contained in a liquid medium according to the invention, the contacting of the liquid food product with the polymer material being carried out for at least 30 minutes, preferably at least 2 hours, and being followed by a step of separating the polymer material sequestering methionine and the liquid food product.

Preferably, the liquid food product is selected from among wines, in particular white wines and rose wines, as well as effervescent wines, dairy products such as milk and its liquid derivatives, malting products, such as beers and ciders, and any other food liquid containing methionine and likely to contain riboflavin, in which a light-struck taste might appear.

As set out hereinbefore, it has been discovered by the present inventors that the elimination, by means of the solid polymer material in accordance with the invention, of methionine molecules present in such liquid products, allows, easily and effectively, avoiding the formation therein of volatile sulfur-containing products responsible for the apparition of a light-struck taste, yet without modifying the organoleptic profile thereof in a detectable manner.

In the context of such an application of the treatment of beverages to prevent the apparition of a light-struck taste, the contact time between the liquid food product and the polymer material may in particular be comprised between 1 day and several months. For example, it may be about 18 months for effervescent wines.

Quite advantageously, in the case of wines matured on their lees and rosé wines, the polymer material may be introduced directly into the maturation barrel, and kept in the latter throughout the alcohol fermentation phase. In the case of effervescent wines, the contact with the polymer material may be performed during sparkling, also in the barrel, and kept throughout this step. In particular, it has been noticed the present inventors that the polymer material according to the invention, and in particular the particular polymer materials as described hereinbefore, may be left in contact with the food liquids, and in particular in alcoholic solutions, for periods as long as several months, for example from 18 to 24 months, yet without being degraded and releasing into the product undesirable residues which cannot be easily separated therefrom.

The method for sequestering methionine molecules contained in a liquid medium according to the invention can also be implemented on any liquid medium containing methionine, in order to remove the latter therefrom, for example with a view to consumption by individuals who are allergic thereto. The method for sequestering methionine molecules contained in a liquid medium may also be applied in any field in which it might be desired to detect the presence of methionine in a liquid, and possibly to assay the methionine contained in a liquid.

Thus, one aspect of the invention relates to a method of detecting the presence of methionine in a liquid, which comprises:
implementing, on a sample of said liquid, a method for sequestering methionine molecules contained in a liquid medium according to the invention, the contacting of the liquid sample with the polymer material being carried out for at least 30 minutes, in particular between 30 minutes and 6 hours, and being followed by a step of separating the polymer material, possibly sequestering methionine, and the liquid sample,
then analyzing the polymer material thus separated for the presence of methionine.

The step of analyzing the polymer material for the presence of methionine may be carried out by any method known to a person skilled in the art. For example, it may be carried out by separation of methionine and the polymer material, in particular as described hereinabove, and analysis of the washing solution, isolated from the solid polymer material, by infrared spectroscopy or proton magnetic nuclear resonance spectroscopy (¹H NMR), in order to detect therein any presence of signals representative of methionine, for example respectively at 2.15 ppm, 2.65 ppm and 3.85 ppm under 300 MHz, D₂O and 298 K conditions.

Optionally, the analysis of the polymer material for the presence of methionine may comprise assaying the methionine contained in this polymer material.

Another aspect of the invention relates to a polymer material suitable for use in a method according to the invention for sequestering methionine molecules contained in a liquid medium.

Such a polymer material can meet one or more of the features described hereinbefore with reference to the description of the method according to the invention.

This polymer material is also capable of sequestering cysteine.

In particular, a polymer material according to the invention may correspond to the formula (V):

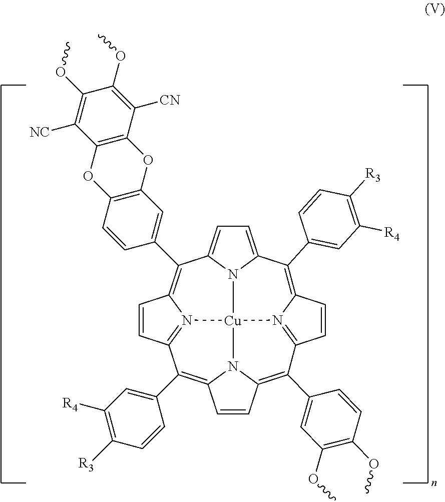

wherein:
R₃ and R4, identical or different, each represent a hydrogen atom or a group selected from among a sulfonate ion or a salt thereof, a carboxylate ion or a salt thereof, an ammonium ion or a salt thereof, or a boronic acid group, the sulfonate ion and salts thereof, in particular an alkali metal salt, for example the sodium salt, being particularly preferred according to the invention,
and n is an integer greater than 2, and preferably less than 1,000.

Another aspect of the invention is a porous solid polymer material obtainable by a synthesis method comprising steps of:
a/ preparing a porphyrin derivative of formula (VI):

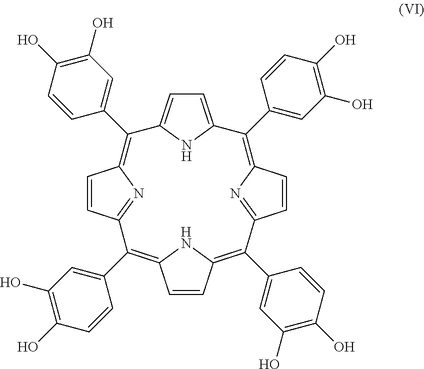 [SMILES: Oc1ccc(-c2c3nc(c(-c4ccc(O)c(O)c4)c4ccc([nH]4)c(-c4ccc(O)c(O)c4)c4nc(c(-c5ccc(O)c(O)c5)c5ccc2[nH]5)C=C4)C=C3)cc1O]

b/ polycondensing said porphyrin derivative of formula (VI) with a compound of formula (VII):

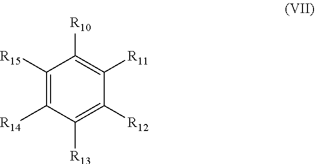

wherein:
R₁₀ and R₁₃ each represent a carbonitrile group —CN,
R₁₁, R₁₂, R₁₄ and R₁₅, identical or different, each represent a halogen atom, in particular a fluorine atom, in the presence of a compound of formula (VIII):

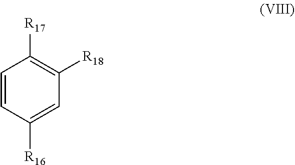

wherein:
R₁₆ represents a hydroxyl, primary or secondary amine or thiol group,
R₁₇ and R₁₈, identical or different, each represent a hydrogen atom or a group selected from among a sulfonate ion or a salt thereof, a carboxylate ion or a salt thereof, an ammonium ion or a salt thereof, or a boronic acid group, the sulfonate ion and salts thereof, preferably an alkali metal salt, for example the sodium salt, being particularly preferred in the context of the invention, so as to form a crosslinked polymer with random branches, and
c/ complexing the porphyrin rings of the polymer obtained in step b/ with copper.

This synthesis method, and the polymer material that it allows obtaining, can meet one or more of the features described hereinbefore with reference to the method according to the invention for sequestering methionine molecules contained in a liquid medium.

In particular:
in formula (VII), R₁₁, R₁₂, R₁₄ and R₁₅ may each represent a fluorine atom,
and/or, in formula (VIII), R₁₆ may represent a hydroxyl group, R₁₇ may represent a sulfonate salt, preferably an alkali metal salt, for example the sodium salt, and R₁₈ may represent a hydrogen atom.

In particular, a porous solid polymer material according to the invention may be represented by the following formula (IXa) illustrating the structure of an average repeat unit of this polymer material:

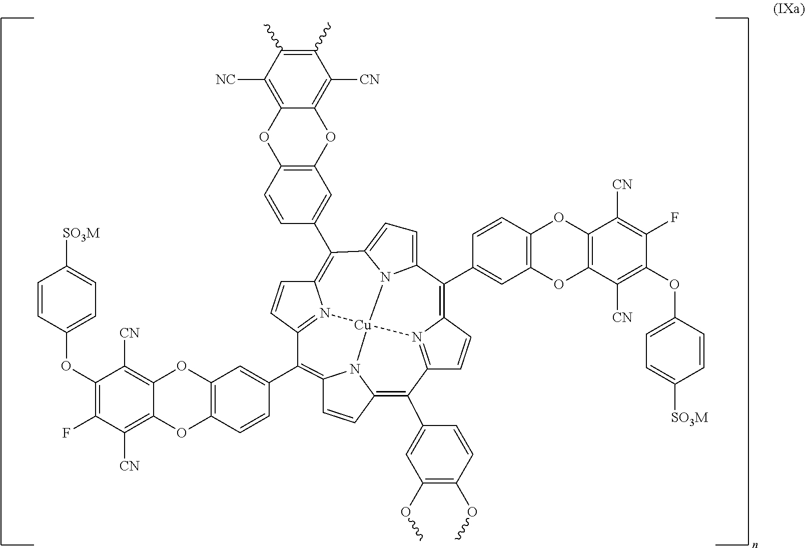

wherein:
M represents an alkali metal, for example sodium, and
n is an integer greater than 2, and preferably less than 1,000.

Another aspect of the invention relates to a method for synthesizing a polymer material according to the invention.

This method has the features described hereinbefore with reference to this polymer material.

### Examples

The features and advantages of the invention will appear more clearly in light of the examples hereinafter, provided simply as a non-limiting illustration of the invention.

### A/ Synthesis of Polymer Materials

Unless indicated otherwise, all of the reagents have been purchased from commercial sources and used without further purification. Pyrrole has been distilled just before use in all cases. The ¹H and ¹³C NMR spectra have been recorded on a Bruker UltraShield 300 MHz spectrometer. The UV-visible spectra have been collected on a PerkinElmer® Lambda® 750 UV/VIS/NIR spectrophotometer.

A.1/ Linear Polymer in Accordance with the Invention—with No Sulfonate Groups (PLI)

The synthesis of this polymer is carried out according to the reaction scheme of FIG. 1, with the exception of the last step which is not carried out.

3-trimethylsilylbromobenzene, of Formula (X):

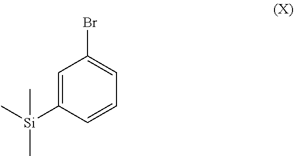

is prepared as follows. 1,3-dibromobenzene (7.12 g, 30.2 mmol) in THF (50 mL) is cooled to −78° C. under argon. n-Butyllithium (1.6 M in hexane, 20.0 mL, 32.6 mmol) is added by syringe and the mixture stirred for 1 h at −78° C. before adding chlorotrimethylsilane (3.61 mL, 33.2 mmol). After stirring for 1.5 h at −78° C., the mixture is brought back to room temperature and stirring is continued overnight. Water (100 mL) is added and the mixture is extracted with diethyl ether (3×50 mL). The organic phases are washed with brine (50 mL), dried over Na₂SO₄, and evaporated under vacuum. The residue is purified by chromatography on silica (eluent: petroleum ether). Yield: 6.27 g (90%).

¹H NMR (300 MHz, 298 K, CDCl₃): δ 7.61 (ddd, J=2.1, 1.1, 0.5 Hz, 1H, He), 7.47 (ddd, J=7.9, 2.1, 1.1 Hz, 1H, Ha), 7.42 (dt, J=7.3, 1.1 Hz, 1H, Hc), 7.25-7.19 (m, 1H, Hb), 0.27 (s, 9H, Hd).

3-trimethylsilylbenzaldehyde, of formula (XI):

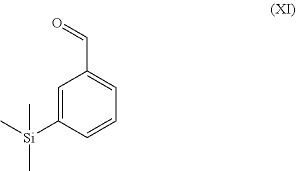

is prepared as follows. 3-bromotrimethylsilylbenzene (X) (6.00 g, 26.2 mmol) is dissolved in THF (90 mL) and cooled to −78° C. under argon. n-butyllithium (1.6 M in hexane, 17.7 mL, 28.3 mmol) is added by syringe and the mixture stirred for 1 h at −78° C. before adding dimethylformamide (3.25 mL, 41.9 mmol). After 1 hour at −78° C., the mixture is brought to room temperature and HCl (1.0 M, 100 mL) added with caution. The mixture is extracted with diethyl ether (3×50 mL). The organic phases are washed with a saturated NaHCO₃ solution (10 mL), brine (50 mL), dried over Na₂SO₄, and evaporated under vacuum. The residue is purified by chromatography on silica (eluent: petroleum ether/diethyl ether). Yield: 4.22 g (91%).

¹H NMR (300 MHz, 298 K, CDCl₃): δ 10.04 (s, 1H, Hf), 8.02 (td, J=1.6, 0.7 Hz, 1H, He), 7.85 (ddd, J=7.6, 1.8, 1.3 Hz, 1H, Ha), 7.78 (dt, J=7.3, 1.3 Hz, 1H, Hc), 7.58-7.46 (m, 1H, Hb), 0.31 (s, 9H, Hd).

5-(3-trimethylsilyl)-dipyrromethane, of formula (XII):

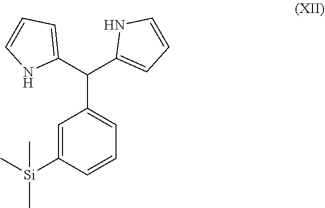 [SMILES: C[Si](C)(C)c1cccc(C(c2ccc[nH]2)c2ccc[nH]2)c1]

is prepared as follows. A solution of 3-trimethylsilylbenzaldehyde (XI) (1.80 g, 10.1 mmol) in pyrrole (52.8 mL, 760 mmol) is de-aerated with argon for 15 min before adding trifluoroacetic acid (77 μL, 1.01 mmol). After stirring in the absence of light for 45 min, triethylamine (0.5 mL) and pyrrole are removed by evaporation under vacuum. The crude product is dissolved in a dichloromethane/triethylamine solution (99:1 v/v) and deposited over silica to be purified by chromatography (silica, eluent: petroleum ether/dichloromethane/triethylamine 66:33:1). Yield: 1.18 g (40%).

¹H NMR (300 MHz, 298 K, CDCl₃): δ 7.94 (s, 2H, Hj), 7.45-7.37 (m, 2H, Hd et He), 7.31 (td, J=7.4, 0.7 Hz, 1H, Hc), 7.17 (dddd, J=7.6, 1.9, 1.3, 0.5 Hz, 1H, Ha), 6.70 (td, J=2.7, 1.6 Hz, 2H, Hi), 6.16 (dt, J=3.4, 2.7 Hz, 2H, Hh), 5.92 (d, J=0.8 Hz, 1H, Hg), 5.47 (s, 1H, Hf), 0.24 (s, 9H, Hb).

3,4-dimethoxybenzene-4-N-tosylimine, of formula (XIII):

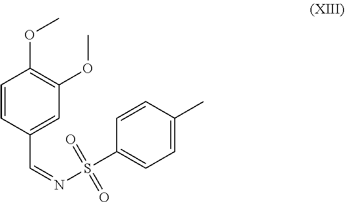 [SMILES: COc1ccc(/C=N\S(=O)(=O)c2ccc(C)cc2)cc1OC]

is prepared as follows. p-toluenesulfonic acid monohydrate (0.622 g, 3.61 mmol) is added to a solution of 3,4-dimethoxybenzaldehyde (6.00 g, 36.1 mmol) and p-toluenesulfonamide (6.18 g, 36.1 mmol) in toluene (100 mL) and the mixture refluxed in a Dean-Stark setup for 3 days. After returning to room temperature, the acid is neutralized by adding triethylamine (3 mL) and the solvent evaporated under vacuum. The product is purified by chromatography on silica (eluent: dichloromethane/petroleum ether 2:) and recrystallized (diethyl ether/ethyl acetate 2: Yield: 7.81 g (68%).

¹H NMR (300 MHz, 298 K, CDCl₃): δ 8.91 (s, 1H, Hf), 7.93-7.83 (m, 2H, Hg), 7.51 (d, J=1.9 Hz, 1H, He), 7.43 (dd, J=8.3, 2.0 Hz, 1H, Hd), 7.38-7.28 (m, 2H, Hh), 6.93 (d, J=8.3 Hz, 1H, Hc), 3.96 (s, 3H, Hb), 3.91 (s, 3H, Ha), 2.43 (s, 3H, Hi).

5,15-di-(3-trimethylsilylphenyl)-10,20-di-(3,4-dimethoxyphenyl) porphyrin, of formula (XIV):

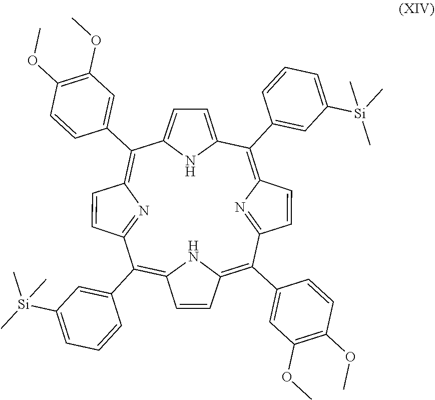

is prepared as follows. 3,4-dimethoxybenzene-4-N-tosylimine (XIII) (1.19 g, 3.74 mmol) and copper (II) triflate (135.0 mg, 0.374 mmol) are dissolved under argon in dichloromethane (150 mL). A solution of 5-(3-trimethylsilylphenyl) dipyrromethane (XII) (1.10 g, 3.74 mmol) in dichloromethane under argon (150 mL) is added thereto in the absence of light. After stirring at room temperature (1.5 h), DDQ (2,3-dichloro-5,6-dicyano-1,4-benzoquinone) (1.70 g, 7.47 mmol) is added to the mixture stirred overnight. Water (50 mL) is added and the mixture is extracted with dichloromethane (3×50 mL). The organic phases are dried over Na₂SO₄, and evaporated under vacuum. The residue is purified by chromatography on silica while collecting the second fraction (eluent: dichloromethane). Yield: 380 mg (23%).

¹H NMR (300 MHz, 298 K, CDCl₃): δ 8.88 (dd, J=19.0, 4.80 Hz, 8H, Hg and Hh), 8.36 (d, J=4.2 Hz, 2H, Hl), 8.19 (m, 2H, Hi), 7.92 (dt, J=7.4, 1.3 Hz, 2H, Hk), 7.75 (m, 6H, Hd, He, and Hj), 7.26 (m, 2H, Hc), 4.18 (s, 6H, Hb), 3.99 (s, 6H, Ha), 0.41 (s, 18H, Hm), −2.73 (s, 2H, Hf) ESI-MS (MeOH): m/z=879.37285 (calc. for C₅₄H₅₅N₄O₄Si₂⁺ 879.37564).

5,15-di-(3-trimethylsilylphenyl)-10,20-di-(3,4-dihydroxyphenyl) porphyrin, of formula (XV):

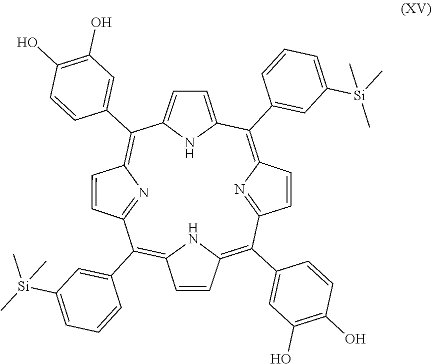

is prepared as follows. 5,15-di-(3-trimethylsilylphenyl)-10,20-di-(3,4-dimethoxyphenyl) porphyrin (XIV) (125 mg, 0.142 mmol) is dissolved in dichloromethane (DCM) (25 mL) under argon and the mixture is cooled to −80° C. Boron tribromide BBr₃ (1.0 M in DCM, 1.14 mmol) is added by syringe and the mixture is stirred at −80° C. for 3 h and then at room temperature overnight. Methanol (10 mL) is added, followed by water (10 mL) and ethyl acetate (30 mL). The aqueous phase is neutralized by adding a saturated aqueous solution of NaHCO₃ and extracted with ethyl acetate (3×20 mL). The organic phases are combined and washed with water (20 mL), brine (20 mL) and then dried over Na₂SO₄. After evaporation of the solvents, the product is recrystallized in acetone/hexane and dried under vacuum. Yield: 153 mg, 81%.

¹H NMR (300 MHz, Acetone) δ 8.92 (dd, J=46.2, 4.9 Hz, 8H, Hg and Hh), 8.37 (s, 2H, Hl), 8.30-8.21 (m, 2H, Hi), 8.02 (dt, J=7.4, 1.2 Hz, 2H, Hk), 7.91-7.80 (m, 2H, Hj), 7.74 (d, J=2.2 Hz, 2H, He), 7.57 (dd, J=8.0, 2.1 Hz, 2H, Hd), 7.27 (d, J=8.0 Hz, 2H, Hc), 0.44 (s, 18H, Hm), −2.71 (s, 2H, Hf) ESI-MS (MeOH): m/z=823.11421 (calc. for C₅₄H₅₅N₄O₄Si₂⁺823.11200).

The polymerization of the porphyrin derivative of formula (XV) is carried out as follows, to form the polymer of formula (XVI):

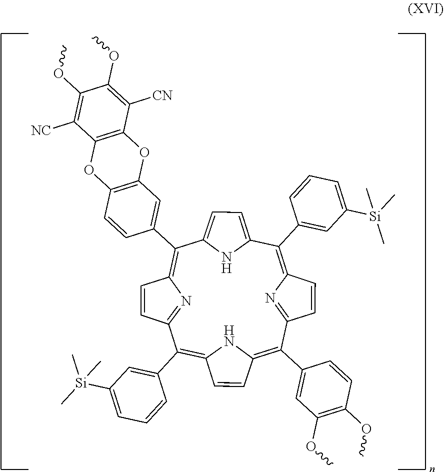

wherein n is an integer comprised between 3 and 999.

5,15-Di-(3-trimethylsilylphenyl)-10,20-di-(3,4-dihydroxyphenyl) porphyrin (0.100 g, 0.121 mmol), and potassium carbonate (0.201 g, 1.46 mmol, 12 eq.) are combined in a round-bottom flask under argon. Anhydrous DMF (12 mL) is added and the mixture brought to 80° C. under stirring. After a few minutes, 2,3,5,6-tetrafluoroterephthalonitrile (0.024 g, 0.121 mmol, 1 eq.) dissolved in DMF (10 mL) is added over 20 min by a syringe. The mixture is refluxed for 2 days, cooled, and diluted with water (100 mL) under ultrasounds for 10 min. The suspension is filtered, washed with water, acetone, methanol, THF, dichloromethane and acetone (2×10 mL). The obtained solid is dried at 55° C. under vacuum. Yield: 104 mg (90%).

The linear polymer PL1 in accordance with the invention, corresponding to the formula (XVII):

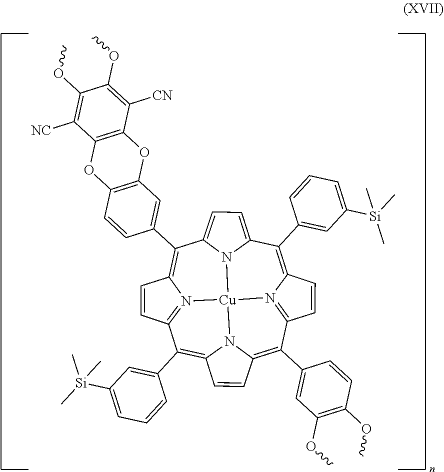

wherein n is an integer comprised between 3 and 999, is obtained by metallation of the polymer of formula (XVI), by suspending the latter (0.060 g) in water (7 ml) and adding Cu(OAc)₂ (0.064 g, 0.318 mmol). The mixture was stirred at 50° C. overnight, cooled, filtered and rinsed with water and then acetone (2×10 ml each), dried at 55° C. under vacuum. Yield: 50 mg (78%).
A.2/ Linear Polymer in Accordance with the Invention—with Sulfonate Groups (PL2)

The polymer PL2 in accordance with the invention, of formula (XVIII):

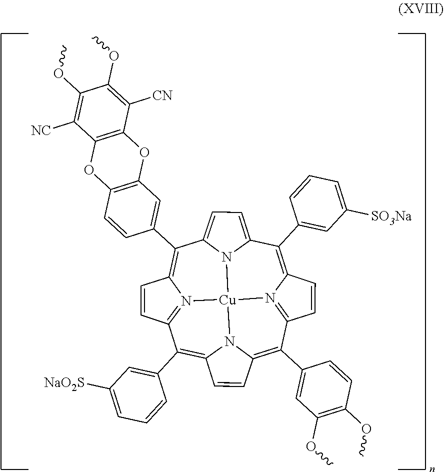

wherein n is an integer comprised between 3 and 999, is prepared from the polymer of formula (XVII), according to the last step of the reaction scheme illustrated in FIG. 1. The latter (0.040 g) is suspended in tetrachloromethane CCl₄ (4 mL) and trimethylsilylchlorosulfonate (0.0386 mL, 0.239 mmol) is added by syringe. The mixture is refluxed for 18 h, cooled, and a solution of NaOH (1 M, 5 mL) and saturated sodium bicarbonate (5 mL) are added. The mixture is stirred vigorously for 1 h and the obtained solid is filtered and washed with water, acetone, dichloromethane and methanol (2×10 mL each) before being dried at 55° C. under vacuum. Yield: 38 mg (90%).

Elemental analysis: expected C, 56.96; H, 2.02; N, 7.67; S, 5.85; Cu, 5.80. found C, 51.35; H, 2.51; N, 7.35; S, 5.77; Cu, 5.70.

A.3/ Crosslinked Polymer not in Accordance with the Invention—with No Copper (Pcomp1)

The synthesis of this polymer is carried out according to the reaction scheme of FIG. 2, with the exception of the last step which is not carried out.

Tetra-(5,10,15,20-(3,4-dimethoxyphenyl)) porphyrin, of formula (XIX):

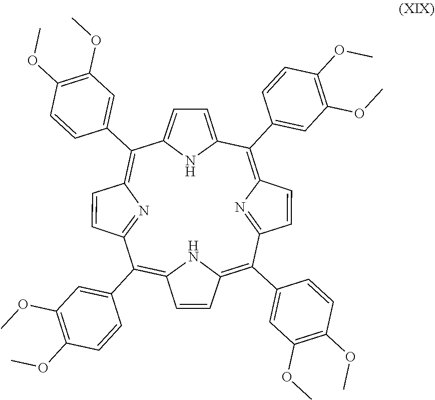

is prepared by adding by dripping pyrrole (0.63 mL, 9.03 mmol) to a solution of 3,4-dimethoxybenzaldehyde (1.50 g, 9.03 mmol) in propionic acid (35 mL) refluxed (at about 170° C.). The mixture is heated with air reflux for 30 min and left to cool at room temperature. The solvent is evaporated under vacuum and the obtained residue purified by chromatography on silica (eluent: dichloromethane) and by re-precipitation in methanol. Yield: 310 mg (15%).

¹H NMR (300 MHz, 298 K, CDCl₃): δ 8.90 (s, 8H, Hf), 7.80-7.74 (m, 8H, Hd and He), 7.31-7.22 (m, 4H, Hc), 4.18 (s, 12H, Hb), 3.99 (s, 12H, Ha), −2.73 (s, 2H, Hg) ESI-MS (MeOH): 855.33620 (calc. for C₅₂H₄₇N₄O₈⁺ 855.33884).

Tetra-(5,10,15,20-(3,4-dihydroxyphenyl)) porphyrin, of formula (XX):

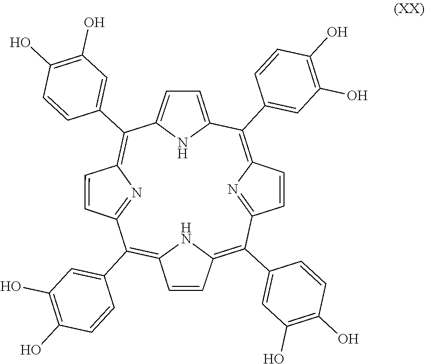 [SMILES: Oc1ccc(-c2c3nc(c(-c4ccc(O)c(O)c4)c4ccc([nH]4)c(-c4ccc(O)c(O)c4)c4nc(c(-c5ccc(O)c(O)c5)c5ccc2[nH]5)C=C4)C=C3)cc1O]

is prepared as follows. A solution of tetra-(5,10,15,20-(3,4-dimethoxyphenyl)) porphyrin (XIX) (300 mg, 0.351 mmol) in a mixture of concentrated HBr hydrogen bromide (20 mL) and glacial acetic acid (15 mL) is refluxed for 3 days. Afterwards, the obtained mixture is cooled and diluted with ethyl acetate (100 mL) and water (50 mL). After neutralization with a saturated NaHCO₃ solution, the mixture is extracted with ethyl acetate (4×50 mL), and the organic phases washed with water, brine, and dried over Na₂SO₄. After evaporation of the volatile phases, the obtained solid is purified by recrystallization (acetone/hexane) to give violet crystals. Yield: 226 mg (86%).

¹H NMR (300 MHz, 298 K, d₆-acetone): δ 8.96 (s, 8H, Hf), 7.74 (d, J=2.1 Hz, 4H, He), 7.56 (dd, J=8.0, 2.1 Hz, 4H, Hd), 7.26 (d, J=8.0 Hz, 4H, Hc), −2.73 (s, 2H, Hg) ESI-MS (MeOH) 743.21308 (calc. for C₄₄H₃₁N₄O₈⁺ 743.21364) UV-Vis (MeOH): λₘₐₓ (nm) (ε (×10³ M⁻¹cm⁻¹)) 422 (275), 518 (13.0), 556 (9.83), 594 (4.89), 650 (5.06).

The crosslinked polymer Pcomp1, wherein the structure of an average monomer unit may be represented by the formula (XXI):

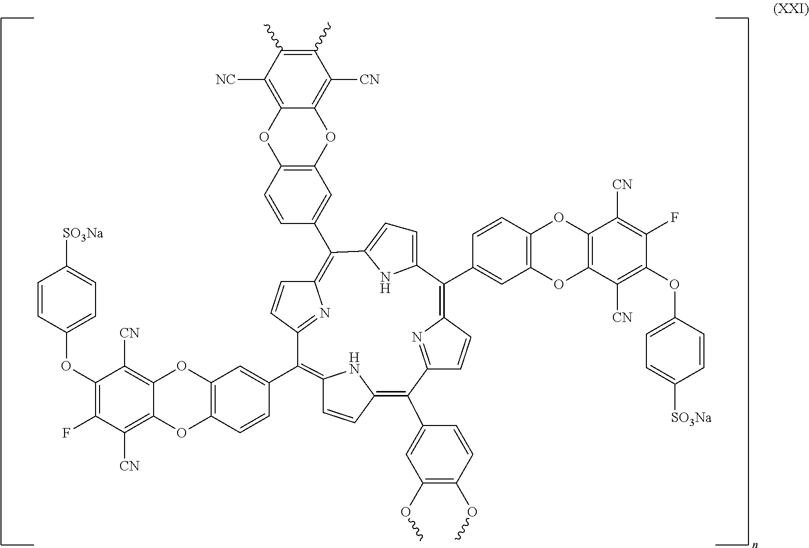

wherein n is an integer comprised between 3 and 999, is obtained by polymerization of the compound of formula (XX). The latter (1.00 g, 1.35 mmol), sodium 4-phenol sulfonate (0.528 g, 2.69 mmol, 2 eq.) and sodium carbonate (4.28 g, 40.4 mmol, 30 eq.) are combined in a round-bottom flask under argon. Anhydrous DMF (50 mL) is added and the mixture brought to 80° C. under stirring. After a few minutes, 2, 3, 5, 6-tetrafluoroterephthalonitrile (0.808 g, 4.04 mmol, 3 eq.) dissolved in DMF (10 mL) is added over 20 min by a syringe. The mixture is refluxed for 2 days, cooled, and diluted with water (100 mL) under ultrasounds for 10 min. The suspension is filtered, washed with water, acetone, methanol, THF, dichloromethane and acetone (2×10 mL). The obtained solid is dried at 55° C. under vacuum. Yield: 1.39 g (67%).
A.4/ Crosslinked Polymer in Accordance with the Invention (PR1)

The crosslinked polymer PR1 is obtained from the polymer Pcomp1 according to the last step of the reaction scheme shown in FIG. 2. It is possible to illustrate the structure of an average monomer unit of this polymer by the formula (XXII):

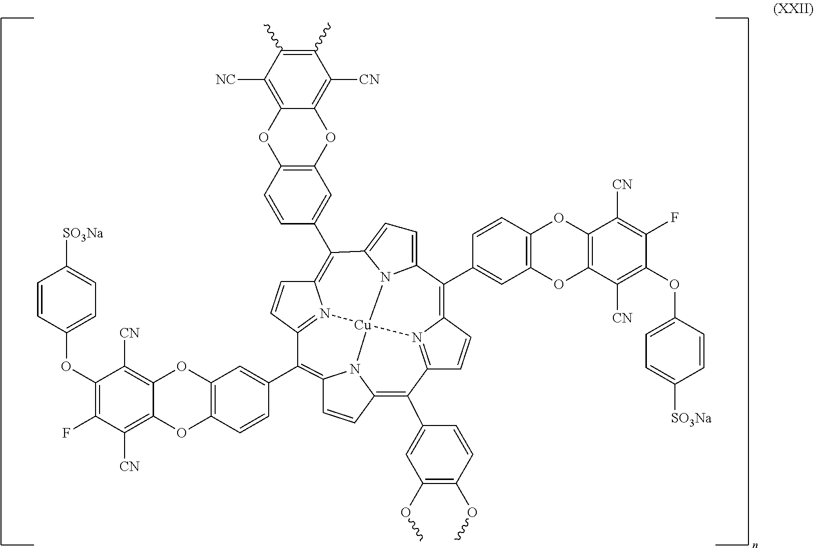

wherein n is an integer comprised between 3 and 999. To this end, the polymer Pcomp1 (1.39 g) is suspended in water (15 mL) and copper (II) diacetate Cu(OAc)₂ (0.927 g, 4.64 mmol) is added. The mixture is stirred for 16 h at 50° C., filtered, washed with water and acetone before being dried at 55° C. under vacuum. Yield: 1.40 g (96%).

Elemental analysis: expected C, 60.18; H, 1.77; N, 8.77; S, 4.02; Cu, 3.98. found C, 56.63; H, 1.88; N, 10.11; S, 1.03; Cu, 6.0.

A.5/ Polymer not in Accordance with the Invention—with No Porphyrin Units (Pcomp2)

A polymer not in accordance with the invention, with no porphyrin units, with monomer units based on a salen ligand containing copper and a crown ether group, known for its capacity to bind to the amine groups of amino acids, is also prepared.

The compound of formula (XXIII):

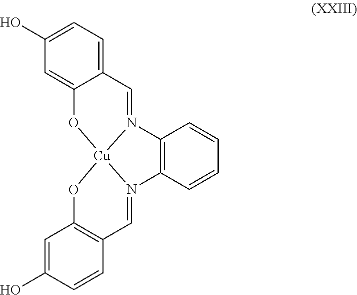

is prepared by adding by dripping a solution of ethanol (10 mL) containing 2,4-dihydroxybenzaldehyde (0.500 g, 3.62 mmol) to a solution of 1,2-diaminobenzene (0.196 g, 1.81 mmol) in ethanol (5 mL). The mixture is refluxed for 2 h and a solution of copper diacetate monohydrate (0.361 g, 1.81 mmol) in ethanol (10 mL) is added slowly. The reflux is stopped after 2 h and the brown precipitate is collected by filtration and washed with cold ethanol (2×10 mL), then acetone (2×10 mL) and dried under vacuum at 70° C. Yield: 670 mg, 90%.

ESI-MS (MeOH): 410.03198 (calc. for C₂₀H₁₅N₂O₄Cu⁺ 410.03223).

The compound of formula (XXIV):

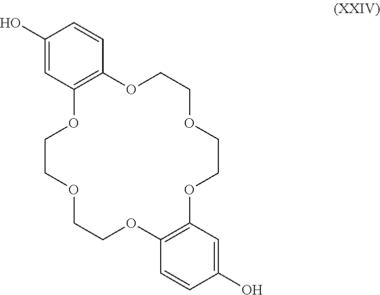 [SMILES: Oc1ccc2c(c1)OCCOCCOc1ccc(O)cc1OCCOCCO2]

is prepared as follows.

Di-(meta-aceto-)benzo-ether-18-crown-6 is first prepared by mixing glacial acetic acid (0.875 g, 15.3 mmol) and an Eaton reagent (29.3 mL, namely 3.3 g, 11.9 mmol of phosphorus pentoxide) and stirring under argon for 15 min. The dibenzo-ether-18-crown-6(2.00 g, 5.10 mmol) is added thereto in one portion and the mixture is stirred at 50° C. overnight before being poured into 150 ml of water and crushed ice. The mixture is extracted with DCM (4×50 mL), and the organic phases are washed with water (2×50 mL) and dried over Na₂SO₄. After evaporation of the solvents, the residue is purified by chromatography on alumina (eluent: chloroform). Yield: 1.50 g, 65%.

¹H NMR (300 MHz, CDCl₃) δ 7.54 (dd, J=8.4, 2.0 Hz, 2H, Hc), 7.49 (d, J=2.0 Hz, 2H, Hd), 6.84 (d, J=8.3 Hz, 2H, Hb), 4.22 (dd, J=5.5, 3.2 Hz, 8H, Hf and Hg), 4.03 (dt, J=8.6, 4.7 Hz, 8H, He and Hh), 2.54 (s, 6H, Ha).

The obtained compound (1.00 g, 2.25 mmol) is dissolved in DCM (15 mL) and hydrated tosylic acid (0.642 g, 3.38 mmol) is added. The mixture is purged with argon for 10 min and metachloroperbenzoic acid m-CBPA (about 50% 3.1 g, 9.00 mmol) is added. The mixture is stirred overnight and filtered on Celite®. The filtrate is washed with an aqueous solution of NaHSO₃ (2×30 mL), water and brine before being dried over Na₂SO₄. After evaporation of the solvents, the obtained product, di-(meta-acetato-)benzo-ether-18-crown-6, is pure at more than 90%. Yield: 550 mg (about 50%).

¹H NMR (300 MHz, CDCl₃, 298 K) δ 6.84 (d, J=9.3 Hz, 2H, Hc), 6.62 (m, 4H, Hb and Hd), 4.14 (m, 8H, He and Hh), 4.01 (m, 8H, Hf and Hg), 2.27 (s, 6H, Ha).

The obtained product (0.570 g, 1.20 mmol) is dissolved in a dichloromethane/methanol mixture (7:11 v/v, 55 mL) using ultrasounds, and the solution is de-aerated. Sodium hydroxide (0.269 g, 6.72 mmol) is dissolved in methanol (8 mL) and de-aerated (argon) before being added and the mixture stirred at room temperature overnight. Concentrated HCl is added until obtaining neutral pH and the white precipitate is collected by filtration. The obtained product of formula (XXIV) is pure at more than 90% and used as such. Yield: 420 mg (about 90%).

¹H NMR (300 MHz, CDCl₃, 298 K) δ 6.78 (d, J=8.7 Hz, 2H), 6.49 (d, J=2.7 Hz, 2H), 6.33 (dd, J=8.7, 2.7 Hz, 2H), 4.18-4.08 (m, 8H, He and Hh), 3.97 (ddt, J=6.7, 4.6, 2.2 Hz, 8H, Hf and Hg).

The polymer Pcomp2, of formula (XXV):

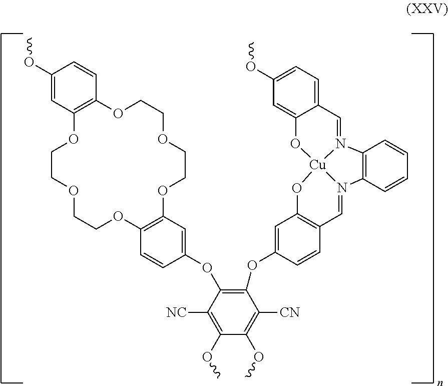

wherein n is an integer comprised between 3 and 999, is prepared as follows.

The compound of formula (XXIII) (40 mg, 0.098 mmol), the compound of formula (XXIV) (42.1 mg, 0.107 mmol) and sodium carbonate (82.7 mg, 0.781 mmol) are combined in a round-bottom flask under argon. Anhydrous DMF (3 mL) is added and the mixture brought to 80° C. under stirring. After a few minutes, 2,3,5,6-tetrafluoroterephthalonitrile (19.5 mg, 0.098 mmol, 1 eq.) dissolved in DMF (10 mL) is added over 20 min by a syringe. The mixture is refluxed for 2 days, cooled, and diluted with water (100 mL) under ultrasounds for 10 min. The suspension is filtered, washed with water, acetone, methanol, THF, dichloromethane and acetone (2×10 mL). The obtained solid is dried at 55° C. under vacuum. Yield: 72 mg (77%).

### B/ Methionine Binding Assays

### B.1/ Study of Methionine Complexation by ¹H NMR

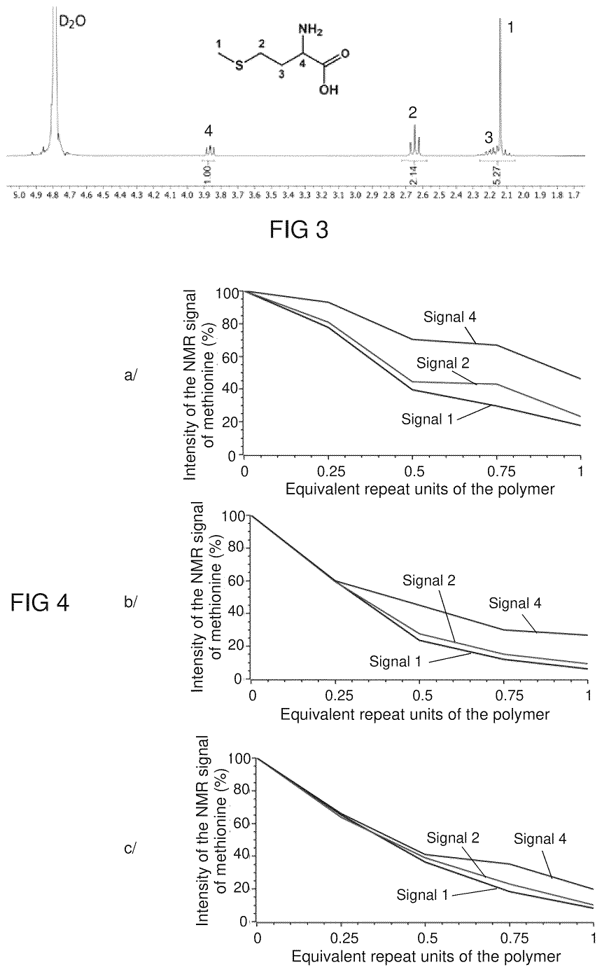

The capacity of the different polymers to absorb methionine has been evaluated by proton nuclear magnetic resonance spectroscopy ¹H NMR. This has been done by dissolving 0.15 mg of methionine in 0.5 ml of D₂O (concentration: 2 mM) and by monitoring the decrease in the intensity of the resonance signals of this amino acid during successive additions of portions of the polymer. The mass of one equivalent of the estimated polymer repeat units has been calculated and 0.25 eq. of the polymer have been added at each addition, until reaching 1.0 eq. This was intended to guarantee that the amount of methionine absorbed by each polymer could be compared by copper ion (one per repeat unit). After each addition of 0.25 eq. of polymer, the NMR tubes have been subjected to ultrasounds for 9 min, the resin has been able to settle at the bottom of the NMR tube for about 5 min, then the NMR spectrum has been recorded. The signal marking system used is shown in FIG. 3.

The results obtained for the signals representative of methionine 1, 2 and 4, as a function of the equivalents of repeat units of the polymer, are shown in FIG. 4, in a/ for the polymer PL1, in b/ for the polymer PL2 and in c/ for the polymer PR1, and in FIG. 5 for the polymer Pcomp1. As regards the polymer Pcomp2, the intensities of the methionine signal having decreased by less than 10% after the addition of 1.0 eq. of the polymer, the curves have been not plotted.

FIG. 4 shows, for each of the tested polymers in accordance with the invention, a significant decrease in the NMR signals of methionine, indicating a significant sequestration of methionine in the polymer. Among the linear polymers, the sequestration is greater for the polymer PL2, in which the porphyrin rings bear sulfonate ion groups (86% decrease on average in the NMR signals at 1 eq.), than for the polymer PL1 which is devoid of these (71% decrease on average in the NMR signals at 1 eq.). The polymer sequestering the largest amount of methionine is the crosslinked polymer PR1 (88% decrease on average in the NMR signals at 1 eq.). This polymer also has the advantages of being easy and quick to synthesize, in only 4 steps, and with a good yield. In comparison, as shown in FIG. 5, the crosslinked polymer with a similar structure but devoid of copper (PComp1) has a much lower methionine sequestration capacity (50% decrease on average in the NMR signals at 1 eq.), which is not enough for many applications. The polymer PComp2, based on salen ligand, copper and 18-crown-6 ether, has an almost zero methionine adsorption capacity (8% decrease on average in the NMR signals at 1 eq.).

B.2/ Isothermal of Association with a Fluorescent Derivative of Methionine in a Model Solution at pH 3.5

A fluorescent derivative of methionine has been prepared by reacting N—BOC-homocysteine (prepared as described in the publication by Mejia, Polym. Chem., 2013, 4, 1969) with 7-allyloxocoumarin (prepared as described in the publication by Orhan, Bioorganic Chemistry, 84, 2019, 355) The N-boc-homocysteine (0.300 g, 1.27 mmol) has been dissolved in dry DMF (8 mL) and the solution purged with argon for 10 min. 7-allyloxocoumarin (0.257 g, 1.27 mmol) and 2,2,2-dimethoxyphenyl-acetophenone (0.065 g, 0.255 mmol) have been added and the mixture has been stirred under irradiation at 365 nm for 16 h. The mixture has been diluted with ethyl acetate (20 mL) and washed with water (30 mL), with brine (20 mL), and then dried over Na₂SO₄, filtered and evaporated under vacuum. The solid thus obtained has been purified by chromatography on silica gel (eluent: 3:1 EtOAc/hexane, then EtOAc, and 9:1 EtOAc/MeOH) to give a yellow solid (176 mg, 31%) which is deprotected by exposure to a solution of HCl (4 M) in dioxane to give the desired product (109 mg, 70%).

¹H NMR (300 MHz, 298 K, DMSO-d₆) δ 7.99 (d, J=9.5 Hz, 1H, Hj), 7.63 (d, J=8.5 Hz, 1H, Hi), 7.02-6.93 (m, 2H, Hg and Hh), 6.29 (d, J=9.5 Hz, 1H, Hk), 4.16 (t, J=6.2 Hz, 2H, Hf), 3.84 (t, J=6.2 Hz, 1H, Ha), 2.74-2.59 (m, 4H, Hc and Hd), 2.10-1.94 (m, 4H, Hb and He)

¹³C NMR (75 MHz, 298 K, DMSO-d6) δ 170.6, 161.7, 158.9, 155.4, 143.7, 130.5, 112.7, 112.5, 112.4, 100.2, 66.4, 52.30, 31.5, 28.6, 27.0, 26.5

ESI-MS (MeOH): 338.1047 (calc. for C₁₆H₂₀NO₅S⁺ 338.1057), 360.0866 (calc. for C₁₆H₁₉NO₅SNa⁺ 360.0876).

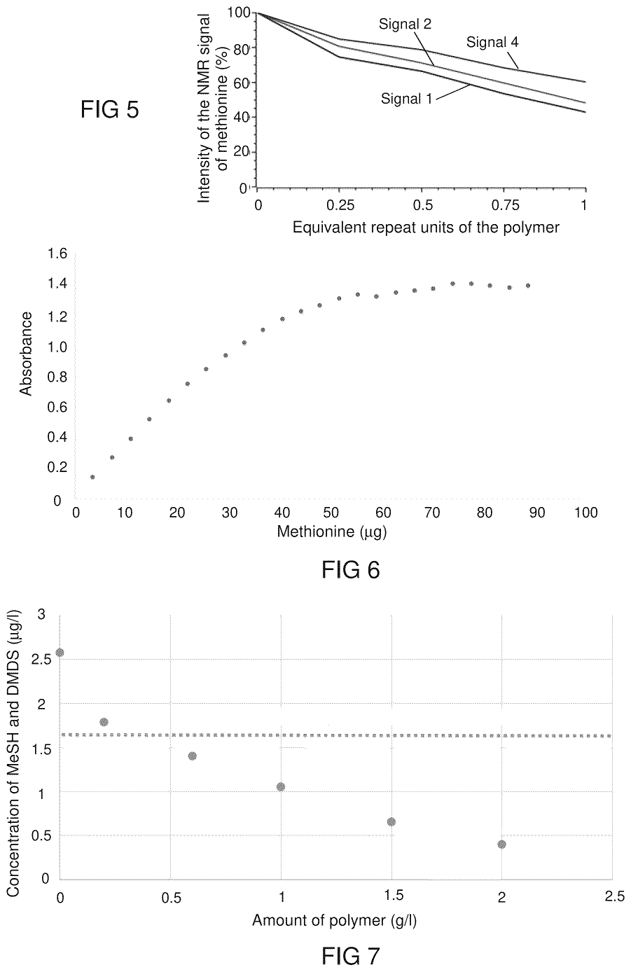

The assay of the affinity of the polymer towards methionine and the measurement of its extraction capacity have been carried out as follows. A quartz cell with a 1 cm optical path has been filled with a solution of water/EtOH (12%) at a pH 3 (2.0 mL) and 0.254 mg of polymer PR1. A concentrated solution of fluorescent methionine (1 mM in the same solution) has been added in 25 μL aliquots. After each addition, the solution of the cell has been mixed for 16 h to reach equilibrium and then left to settle before determining the concentration of free methionine by electronic absorption at 324 nm (ε₃₂₄=10,800 M⁻¹cm⁻¹). The curve representing the measured absorbance as a function of the concentration of methionine added in the cell thus obtained is shown in FIG. 6.

Afterwards, the concentration of free methionine has been analyzed by the Scatchard technique by reporting the ratio ([complexed methionine]/[free methionine]) as a function of the concentration of complexed methionine. By linear regression, a concentration of complexing sites of 248 mg of methionine per gram of PR1 polymer (79% of the initial amount of methionine has been absorbed by the polymer, has been found. This corresponds to 1.0 mg of polymer binding 0.298 mg of Met*at concentrations close to 1×10⁻⁴ M, the approximate concentration of methionine in champagne). Two distinct complexation sites having association constants of 7.3×10⁶ M⁻¹ and 3.8×10⁵ M⁻¹ have been observed. An average association constant weighted by the number of sites equal to 3.0×10⁶ M⁻¹ is deduced therefrom.

The same experiment, carried out with the linear polymer PL2, leads to the adsorption of 89% of the initial fluorescent methionine by the polymer.

### C/ Protection in a Model Solution Subjected to Irradiation—Elimination of the Volatile Sulfur-Containing Species

A model solution of synthetic wine has been prepared by dissolving 3.5 g of tartaric acid in 1 L of a water 88%/ethanol 12% mixture in vol % and the pH has been adjusted to 3.5 using an aqueous 1 M sodium hydroxide solution. Riboflavin (riboflavin concentration of 0.05 mM) and methionine (0.1 mM) have then been dissolved therein. This solution has been stirred for 1 week using an orbital stirrer in the presence of an amount of 0.2 g/l of crosslinked polymer in accordance with the invention PR1. Afterwards, the polymer material has been filtered and the obtained solution has been placed in two transparent funnels (2×4.5 ml) which have been sealed and irradiated for 5 min at 250 W/m². Afterwards, the funnels have been opened and the obtained solution has been analyzed by micro-extraction on solid phase coupled with gas-phase chromatography with detection by pulse flame photometry (SPME-GC-PFPD) in split 200, to detect the following species: methanethiol, dimethyldisulfide (DMDS) and dimethyltrisulfide (DMTS). In parallel, a control sample without resin has been processed in the same manner. The obtained results are shown in Table 1.

TABLE 1

| | With no polymer | With polymer PR1 |
|---|---|---|
| Methanethiol (μg/l) | 184 | Not detected |
| DMDS (μg/l) | 276 | 10.1 |
| DMTS (μg/l) | 133 | Not detected |

These results clearly indicate that the polymer in accordance with the invention has allowed reducing, in a particularly considerable proportion, the concentrations of volatile sulfur-containing species forming in the model solution following exposure to light: this decrease is 96% for DMDS. Each of the methanethiol and the DMTS is in an amount lower than its detection threshold. These results demonstrate that the polymer material has a sufficient methionine adsorption capacity to almost completely prevent the apparition of volatile sulfur-containing products responsible for the light-struck taste.

### D/ Treatment of an Effervescent Wine

The same protocol as that described in section C/hereinabove has been applied to effervescent wine corresponding to the registered designation of origin Champagne, freshly open, and by using variable amounts of the polymer material PR1.

Seven samples have been stirred for 1 week: 5 samples intended to be irradiated correspond to amounts of polymer varying from 0.2 g/l to 2 g/l, 1 sample corresponds to a control without polymer intended to be irradiated and 1 sample corresponds to a control without polymer which will not be irradiated.

The results obtained are shown in FIG. 7, in terms of total concentration of methanethiol (MeSH) and dimethyl disulfide (DMDS) as a function of the amount of polymer used. Moreover, after irradiation, this effervescent wine has been subjected to a sensory analysis by a trained sensory panel. The score of 4.2/10 has been attributed thereto on the light-struck taste criterion. The threshold of perception of a light-struck taste by the sensory panel on this sample of the irradiated wine not in contact with the polymer is illustrated by a dotted line in the figure. As one can clearly see, the polymer in accordance with the invention has been effective for preventing the apparition of MeSH and DMDS starting from concentrations higher than or equal to 0.2 g/L. Above this concentration, the score attributed by the sensory panel, on the light-struck taste criterion, should thus be greater than 4.2/10.

With regards to the irradiated control without polymer, for the irradiated sample containing 2 g/l of polymer material, a reduction of 84% in the concentration of volatile sulfur-containing species MeSH and DMDS is observed. This demonstrates that enough methionine has been sequestered by the polymer material in accordance with the invention to almost completely prevent the apparition of substances responsible for the light-struck taste.

### E/ Competitiveness

To evaluate the selectivity of the polymer PR1 with respect to methionine relative to proline, another amino acid present in a large amount in effervescent wines, solutions containing equal concentrations of methionine and proline (2 mM in 0.5 ml of D₂O) have been prepared. 0.4 mg of polymer PR1 have then been added to each solution and the reduction in the intensity of the ¹H NMR signals of the two amino acids have been recorded. An average decrease in the signals of 41% is obtained for methionine. The average decrease in the signals relating to proline is much lower, equal to 19%.

### F/ Recyclability

Taking the contents of the cuvette of the experiment described in point B.2/ hereinabove, it has been attempted to remove the fluorescent methionine Met*from the polymer PR1 under alkaline conditions, so that the binding capacity could be measured again so as to determine the recyclability of the material. The procedure has been as follows: the contents of the cuvette (0.5 mg of polymer having reached the recovery equilibrium of Met*) has been transferred into a centrifuge tube, by rinsing the cuvette with water (MilliQ, pH 10 by addition of NaOH). The suspension has been subjected to ultrasounds for 10 min, centrifuged (5,000 rpm, 8 min) and the supernatant has been carefully discarded without breaking the pellet. The resin has been washed with water (10 ml, MilliQ, pH 10 by addition of NaOH), sonicated, centrifuged and the supernatant discarded 5 times more. Finally, the resin has been rinsed with water (2 ml, pH=3.0) to ensure that no NaOH residue remained, centrifuged and the supernatant has been discarded. The resin has been resuspended in water (2.5 ml, pH=3.0) and transferred into a cuvette. The UV-visible spectrum has been recorded. The absorbance measured at 324 nm corresponded to a concentration of Met*of 7% of the initially added amount.

At this stage, Met*(0.5 ml, 1 mM solution in water at pH=3.0) has been added (concentration of Met*=1.67×10⁴ M⁻¹). The mixture has been stirred overnight to enable it to reach equilibrium, and the UV-visible spectrum has been recorded again. The absorbance measured at 324 nm corresponded to 45% of the Met*which has been added the second time, the remainder having therefore been bound by the polymer. This demonstrates that the polymer in accordance with the invention can be recycled by washing, the latter restoring its methionine adsorption capacity.

### G/ Stability for Food Contact

Studies have been carried out to quantify the amount of porphyrin and copper released in a wine model solution (water 88%, ethanol 12%, tartaric acid to obtain a pH of 3.5) after being brought into contact with the polymer according to the invention PR1 for 10 days at 60° C. (accelerated aging conditions).

2.0 mg of polymer have been suspended in 3.0 ml of the model solution and left at rest at room temperature for one day. The same experiment has been carried out but with the solution heated at 60° C. for 10 days. The solutions have been filtered through a syringe filter and the UV-visible spectra of each have been recorded. Porphyrins have a very high absorption at about 420 nm and would be easily detected if a porphyrin species had been dissolved. For each of the experiments, no signal is detected at 420 nm.

To detect whether copper ions would escape from the polymer material under the same conditions, the solution of the experiment hereinabove left at 60° C. for 10 days has been stirred vigorously for 10 min with 5 ml of a DCM solution (7×10⁻⁷ M) of dithizone. Dithizone is an organic ligand which strongly absorbs around 607 nm in the visible spectrum. Upon complexation of the Cu(II) ions, a new maximum is observed at about 540 nm. The spectrum obtained after exposure of the dithizone solution to the aqueous solution which has been in contact with the polymer PR1 clearly shows, compared to the spectrum of dithizone alone, a new maximum towards 540 nm, corresponding to the formation of the complex of dithizone with Cu(II).

It arises from this spectrum that about half of the added total dithizone has been complexed with the Cu(II) ions. This corresponds to about 0.01% of the total amount of Cu(II) ions supposed to be present in the mass of the used polymer PR1: no significant release of copper has thus been observed.

It arises from these experiments that the polymer material PR1 according to the invention has not released its porphyrin and cupric ion components in the model solution, and that it is compatible with use in contact with food.

What is claimed is:

1. A method for sequestering methionine molecules contained in a liquid medium, comprising contacting said liquid medium with a solid polymer material comprising monomer units based on porphyrin containing copper.

2. The method according to claim 1, wherein said polymer material contains at least one hydrophilic group.

3. The method according to claim 1, wherein at least one of said monomer units has the general formula (I): 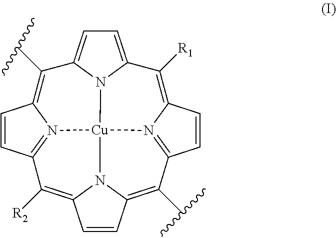
wherein R₁ and R₂, identical or different, each represent a hydrogen atom or a group of formula (II): 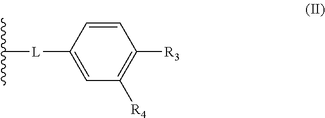
wherein:
R₃ and R₄, identical or different, each represent a hydrogen atom or a group selected the group consisting of a sulfonate ion or a salt thereof, a carboxylate ion or a salt thereof, an ammonium ion or a salt thereof, and a boronic acid group,
and wherein L represents a covalent bond or a spacer arm.

4. The method according to claim 3, wherein, in formula (II), L represents a spacer arm of formula (III): 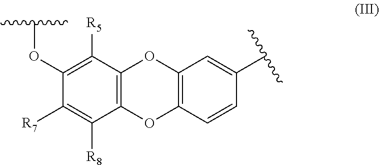
wherein:
R₅ and R₆ each represent a carbonitrile group —CN,
and R₇ represents a halogen atom.

5. The method according to claim 1, wherein said monomer units are connected by a bonding group of formula (IV): 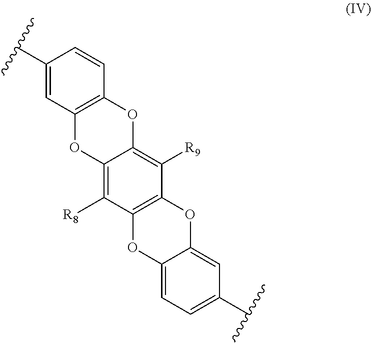
wherein R₃ and R₉ each represent a carbonitrile group —CN.

6. The method according to claim 1, wherein said polymer is a linear polymer.

7. The method according to claim 6, wherein said polymer has the formula (V): 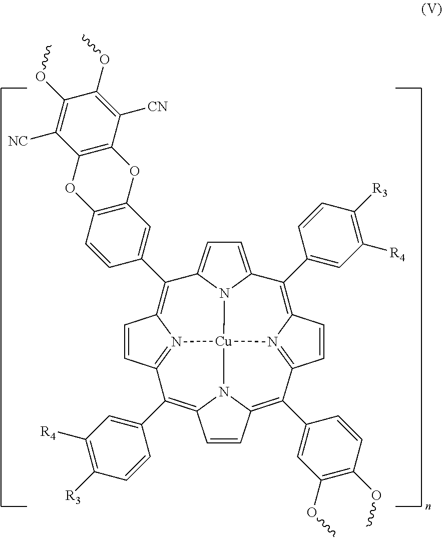
wherein:
R₃ and R₄, identical or different, each represent a hydrogen atom or a group selected from the group consisting of a sulfonate ion or a salt thereof, a carboxylate ion or a salt thereof, an ammonium ion or a salt thereof, and a boronic acid group,
and n is an integer greater than 2.

8. The method according to claim 7, wherein, in formula (V), R₃ represents a hydrogen atom and R₄ represents a sulfonate ion or a salt thereof.

9. The method according to claim 1, wherein said polymer is a crosslinked polymer.

10. The method according to claim 9, wherein said polymer is obtainable by a synthesis method comprising the steps of:
a/ preparing a porphyrin derivative of formula (VI): 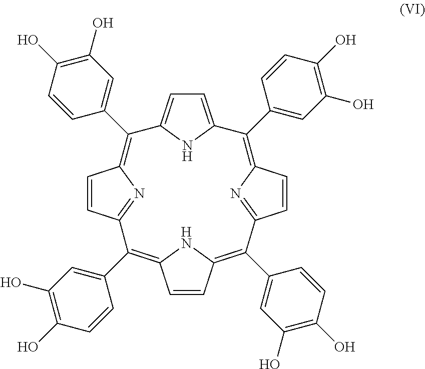 [SMILES: Oc1ccc(-c2c3nc(c(-c4ccc(O)c(O)c4)c4ccc([nH]4)c(-c4ccc(O)c(O)c4)c4nc(c(-c5ccc(O)c(O)c5)c5ccc2[nH]5)C=C4)C=C3)cc1O]
b/ polycondensing said porphyrin derivative of formula (VI) with a compound of formula (VII): 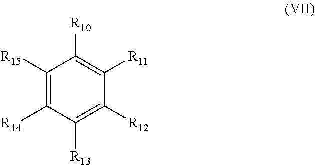
wherein:
R₁₀ and R₁₃ each represent a carbonitrile group —CN,
R₁₁, R₁₂, R₁₄ and R₁₅, identical or different, each represent a halogen atom,
in the presence of a compound of formula (VIII): 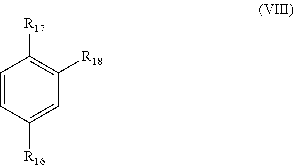
wherein:
R₁₆ represents a hydroxyl, primary or secondary amine or thiol group,
R₁₇ and R₁₈, identical or different, each represent a hydrogen atom or a group selected from the group consisting of a sulfonate ion or a salt thereof, a carboxylate ion or a salt thereof, an ammonium ion or a salt thereof, and a boronic acid group, and
c/ complexing the porphyrin rings of the polymer obtained in step b/ with copper.

11. The method according to claim 10, wherein, in formula (VIII), R₁₆ represents a hydroxyl group, R₁₇ represents a sulfonate salt and R₁₈ represents a hydrogen atom.

12. The method according to claim 1, wherein said polymer material is introduced into said liquid medium at a dose comprised between 0.01 and 50 g/l.

13. The method according to claim 1, comprising a step of separating said liquid medium and the polymer material sequestering methionine.

14. The method according to claim 13, comprising a final step of separating the methionine molecules from said polymer material.

15. A method of treating a liquid food product to reduce the risk of a light-struck taste occurring therein, comprising implementing on said liquid food product a method for sequestering methionine molecules contained in a liquid medium according to claim 1, the contacting of said liquid food product with the polymer material being carried out for at least 30 minutes, and being followed by a step of separating said polymer material and said liquid food product.

16. The method of treating a food liquid according to claim 15, wherein said liquid food product is selected from the group consisting of wines, dairy products and malting products.

17. A method of detecting the presence of methionine in a liquid, comprising:
implementing, on a sample of said liquid, a method for sequestering methionine molecules contained in a liquid medium according to claim 1, the contacting of said liquid sample with the polymer material being carried out for at least 30 minutes, and being followed by a step of separating said polymer material and said liquid sample,
then analyzing said polymer material for the presence of methionine.

18. A porous solid polymer material obtainable by a synthesis method comprising the steps of:
a/ preparing a porphyrin derivative of formula (VI): 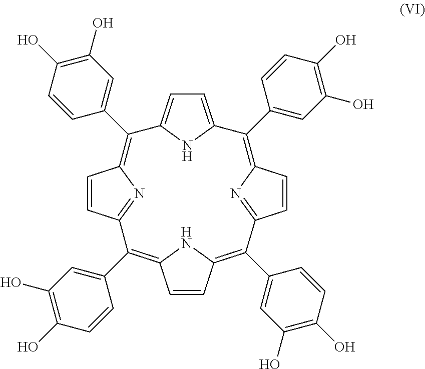 [SMILES: Oc1ccc(-c2c3nc(c(-c4ccc(O)c(O)c4)c4ccc([nH]4)c(-c4ccc(O)c(O)c4)c4nc(c(-c5ccc(O)c(O)c5)c5ccc2[nH]5)C=C4)C=C3)cc1O]
b/ polycondensing said porphyrin derivative of formula (VI) with a compound of formula (VII): 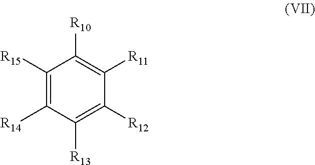
wherein:
R₁₀ and R₁₃ each represent a carbonitrile group —CN,
R₁₁, R₁₂, R₁₄ and R₁₅, identical or different, each represent a halogen atom,
in the presence of a compound of formula (VIII): 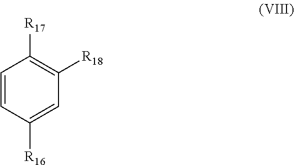
wherein:
R₁₆ represents a hydroxyl, primary or secondary amine or thiol group,
R₁₇ and R₁₃, identical or different, each represent a hydrogen atom or a group selected from the group consisting of a sulfonate ion or a salt thereof, a carboxylate ion or a salt thereof, an ammonium ion or a salt thereof, and a boronic acid group, and
c/ complexing the porphyrin rings of the polymer obtained in step b/ with copper.

19. The material according to claim 18, wherein, in formula (VIII), R₁₆ represents a hydroxyl group, R₁₇ represents a sulfonate salt, and R₁₈ represents a hydrogen atom.

20. The method according to claim 2, wherein said polymer material contains at least one sulfonate ion group or a salt thereof.
